(19) United States
(12) Patent Application Publication
(10) Pub. No.: US 2011/0301235 A1
(43) Pub. Date: Dec. 8, 2011

(54) ORGANOSELENIUM COMPOUNDS AND USES THEREOF

(75) Inventors: Daniel A. Erlanson, San Francisco, CA (US); Stig K. Hansen, Kensington, CA (US)

(73) Assignee: Alquest Therapeutics, Inc., Kensington, CA (US)

(21) Appl. No.: 12/959,285

(22) Filed: Dec. 2, 2010

Related U.S. Application Data
(60) Provisional application No. 61/266,086, filed on Dec. 2, 2009.

(51) Int. Cl. A61K 31/165; A61K 31/197; A61K 31/24; A61K 31/167; A61K 31/185; A61P 25/28; A61P 3/04; A61P 17/00; A61P 11/00; A61P 25/00; A61P 9/10; A61P 27/02; C07C 391/02; A61P 25/30

(52) U.S. Cl. 514/533; 564/154; 562/426; 560/16; 564/162; 562/44; 514/616; 514/562; 514/539; 514/618; 514/576

(57) ABSTRACT

Provided herein are organoselenium compounds and pharmaceutical compositions thereof. Also provided herein are methods of treatment, prevention, or amelioration of a variety of medical disorders using the compounds and pharmaceutical compositions provided herein.

This application claims the benefit under 35 U.S.C. §119(e) from U.S. Provisional Application Ser. No. 61/266,086, filed Dec. 2, 2009, which is herein incorporated by reference in its entirety.

### FIELD

Provided herein are organoselenium compounds and pharmaceutical compositions thereof. Also provided herein are methods of treatment, prevention, or amelioration of a variety of medical disorders using the compounds and pharmaceutical compositions provided herein.

### BACKGROUND

Reactive oxygen and nitrogen species occur during natural metabolism and the immune system produces these transient intermediates intentionally to remove foreign material such as, for example, bacteria and viruses. Although certain levels of reactive species can be tolerated in vivo, excessive amounts cause severe damage to cells and tissues of the human body. Compounds that act as antioxidants, such as for example, Vitamin C, Vitamin E and selenium can reduce the levels of reactive species in vivo and are recommended by the FDA as useful supplements for daily consumption.

The pathogenesis of numerous diseases, including, for example, cancer, metabolic (e.g., obesity, diabetes, hypertension, metabolic syndrome, nephropathy, neuropathy or atherosclerosis), inflammatory (e.g., psoriasis, irritable bowel disease, sunburn, cystic fibrosis or arthritis) and neurodegenerative diseases (e.g., Parkinson's disease, Alzheimer's disease, Parkinson's disease, Huntington's disease and multiple sclerosis) involve reactive oxygen and nitrogen species produced by mitochondrial dysfunction and cellular enzymes, including, but not limited to, Nox-family enzymes, xanthine oxidases, or NADH oxidases and exposure to environmental toxins. However, current therapeutic antioxidants lack satisfactory pharmacological profiles to effectively manage these diseases.

Thus, there is a continuing need for less toxic, more effective pharmaceutical agents to treat, prevent or ameliorate a variety of disorders associated with excessive formation of reactive oxygen and nitrogen species.

### SUMMARY

Provide herein are organoselenium compounds that satisfy these and other needs. Also provided herein are compositions and methods of using the compounds and compositions for the treatment, prevention, or amelioration of various diseases.

In some embodiments, compounds of Formula (I):

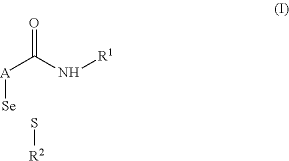

or salts, solvates or hydrates thereof are provided, wherein:
A is aryl, substituted aryl, heteroaryl or substituted heteroaryl;
R¹ is alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R² is alkyl, substituted alkyl, heteroalkyl, substituted heteroalkyl, aryl, substituted aryl, arylalkyl, substituted arylalkyl, heteroaryl, substituted heteroaryl, heteroarylalkyl, substituted heteroarylalkyl,

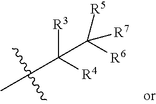

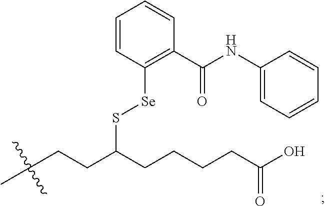

R³ is hydrogen, —OR⁹, —S(O)ₖR¹⁰, —NR¹¹R¹², —NR¹⁰COR¹³, —CO₂R¹³, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R⁵ is hydrogen, —OR¹⁴, —S(O)ₗR¹⁵, —NR¹⁶R¹⁷, —NR¹⁵COR¹⁸, —CO₂R¹⁸, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R⁷ is —CO₂R¹⁹, —CONR²⁰R²¹, —NR²²R²³, —NR²⁴COR¹⁹ or —S(O)ₘR²⁴;
R⁴, R⁶ and R⁹-R²⁴ are independently hydrogen, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl; and
k, l and m are independently 0, 1 or 2;
provided that when A and R¹ are phenyl substituted with natural amounts of deuterium that R² is not

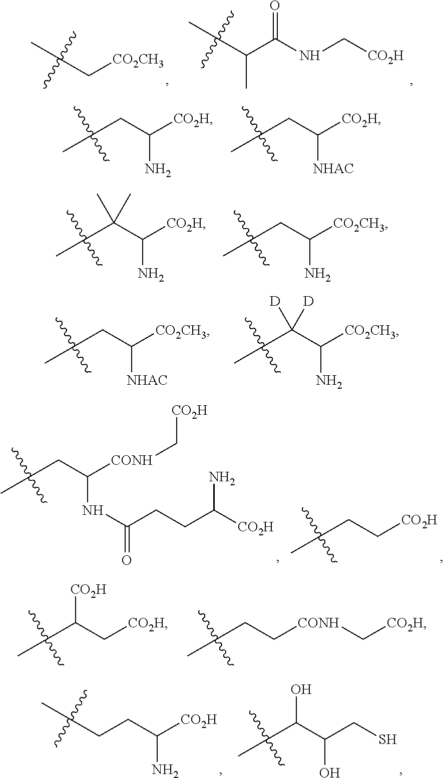

ethyl, allyl, n-propyl, isopropyl, t-butyl, n-butyl, n-hepyl, n-octyl, cyclohexyl, 4-trans-t-butylcyclohexyl, 4-cis-t-butylcyclohexyl, phenyl, p-methylphenyl, p-chlorophenyl, o-benzoic acid, o-methyl benzoate, 2-napthyl, benzyl, 2-pyridyl,

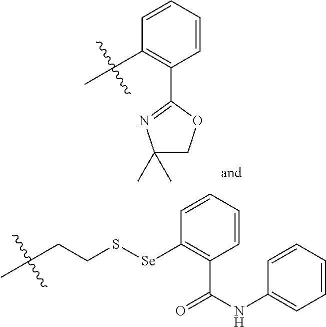

wherein the above compounds are substituted with natural amounts of deuterium, except where deuterium is explicitly specified in the structure of the above compounds.

In other embodiments, compounds of Formula (II):

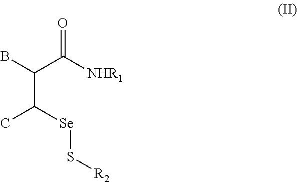

or salts, solvates or hydrates thereof are provided, wherein:
B and C together with the atoms to which they are bonded form an aryl, substituted aryl, heteroaryl or substituted heteroaryl ring;
R¹ is alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R² is alkyl, substituted alkyl, heteroalkyl, substituted heteroalkyl, aryl, substituted aryl, arylalkyl, substituted arylalkyl, heteroaryl, substituted heteroaryl, heteroarylalkyl, substituted heteroarylalkyl,

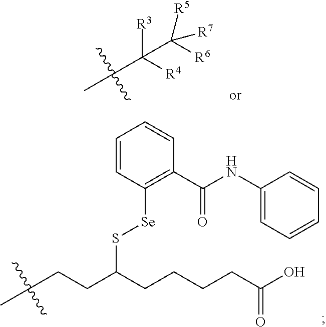

R³ is hydrogen, —OR⁹, —S(O)ₖR¹⁰, —NR¹¹R¹², —NR¹⁰COR¹³, —CO₂R¹³, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R⁵ is hydrogen, —OR¹⁴, —SO(O)ₗR¹⁵, —NR¹⁶R¹⁷, —NR¹⁵COR¹⁸, —CO₂R¹⁸, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R⁷ is —CO₂R¹⁹, —CONR²⁰R²¹, —NR²²R²³, —NR²⁴COR¹⁹ or —S(O)ₘR²⁴;
R⁴, R⁶ and R⁹-R²⁴ are independently hydrogen, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl; and
k, l and m are independently 0, 1 or 2;
provided that when A and R¹ are phenyl substituted with natural amounts of deuterium that R² is not

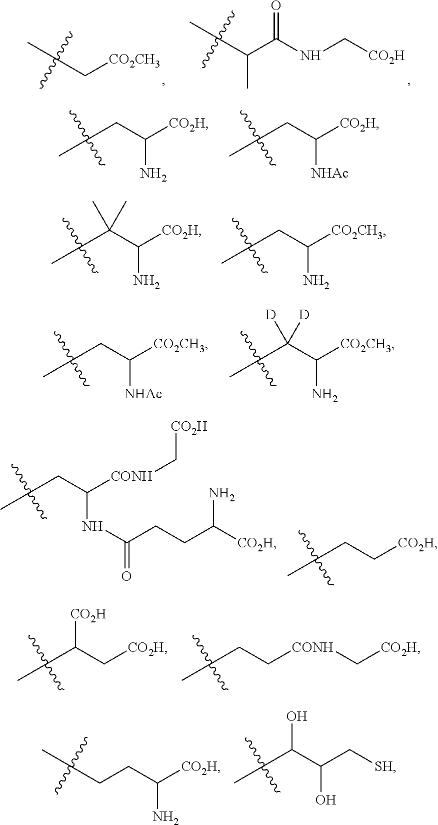

ethyl, allyl, n-propyl, isopropyl, t-butyl, n-butyl, n-hepyl, n-octyl, cyclohexyl, 4-trans-t-butylcyclohexyl, 4-cis-t-butylcyclohexyl, phenyl, p-methylphenyl, p-chlorophenyl, o-benzoic acid, o-methyl benzoate, 2-napthyl, benzyl, 2-pyridyl,

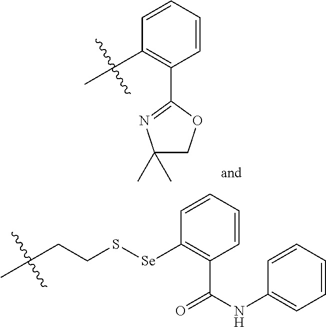

wherein the above compounds are substituted with natural amounts of deuterium, except where deuterium is explicitly specified in the structure of the above compounds.

In still other embodiments, compounds of Formula (III)

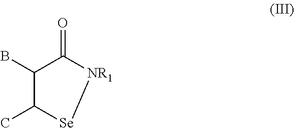

or salts, solvates or hydrates thereof are provided, wherein:
B and C together with the atoms to which they are bonded form an aryl, substituted aryl, heteroaryl or substituted heteroaryl ring;
R¹ is alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl wherein at least one hydrogen in Formula (III) is substituted with deuterium.

Also provided are derivatives, including prodrugs of the compounds described herein. Further provided are pharmaceutical compositions containing the compounds provided herein and a vehicle.

Methods of treating, preventing, or ameliorating one or more symptoms of a wide variety of medical disorders, including but not limited to, cancer, metabolic (e.g., obesity, diabetes, hypertension, metabolic syndrome, nephropathy, neuropathy or atherosclerosis), inflammatory (e.g., psoriasis, irritable bowel disease, sunburn, cystic fibrosis or arthritis) addictive disorders, skin disorders, lung disorders or respiratory diseases, neuropsychiatric disorders, traumatic injury, ischemia, ischemia-reperfusion injury, ocular disease and neurodegenerative diseases (e.g., Parkinson's disease, Alzheimer's disease, Parkinson's disease, Huntington's disease, Pick's disease, amytrophic lateral scleosis, prion diseases (e.g. Creutzfeldt-Jakob disease, Gerstmann-Straussler-Scheinker syndrome, fatal familial insomnia, Kuru, scrapie, or chronic wasting disease) and multiple sclerosis) using the compounds and compositions described herein are provided. In some embodiments, the compounds of Formula (I) and (II) when used to treat, prevent, or ameliorate one or more symptoms of a wide variety of medical disorders are not limited by the proviso clause, supra. In practicing the methods, effective amounts of the compounds or compositions containing therapeutically effective concentrations of the compounds are administered.

### BRIEF DESCRIPTION OF THE DRAWINGS

FIG. 1 illustrates oxidation of the fluoroprobe as detected by monitoring the increase in fluorescence with time.

### DETAILED DESCRIPTION

### Definitions

Unless defined otherwise, all technical and scientific terms used herein have the same meaning as is commonly understood by one of ordinary skill in the art to which this invention belongs. All patents, applications, published applications and other publications are incorporated by reference in their entirety. In the event that there are a plurality of definitions for a term herein, those in this section prevail unless stated otherwise.

“Alkyl,” by itself or as part of another substituent, refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. In some embodiments, an alkyl group comprises from 1 to 20 carbon atoms (C₁-C₂₀ alkyl). In other embodiments, an alkyl group comprises from 1 to 10 carbon atoms (C₁-C₁₀ alkyl). In still other embodiments, an alkyl group comprises from 1 to 6 carbon atoms (C₁-C₆ alkyl).

“Alkanyl,” by itself or as part of another substituent, refers to a saturated branched, straight-chain or cyclic alkyl radical derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkenyl,” by itself or as part of another substituent, refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl,” by itself or as part of another substituent refers to an unsaturated branched, straight-chain or cyclic alkyl radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Alkoxy,” by itself or as part of another substituent, refers to a radical of the formula —O—R⁴⁰⁰, where R⁴⁰⁰ is alkyl or substituted alkyl as defined herein.

“Acyl” by itself or as part of another substituent refers to a radical —C(O)R⁴⁰¹, where R⁴⁰¹ is hydrogen, alkyl, substituted alkyl, aryl, substituted aryl, arylalkyl, substituted arylalkyl, heteroalkyl, substituted heteroalkyl, heteroarylalkyl or substituted heteroarylalkyl as defined herein. Representative examples include, but are not limited to formyl, acetyl, cyclohexylcarbonyl, cyclohexylmethylcarbonyl, benzoyl, benzylcarbonyl and the like.

“Aryl,” by itself or as part of another substituent, refers to a monovalent aromatic hydrocarbon group derived by the removal of one hydrogen atom from a single carbon atom of a parent aromatic ring system, as defined herein. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In some embodiments, an aryl group comprises from 6 to 20 carbon atoms (C₆-C₂₀ aryl). In other embodiments, an aryl group comprises from 6 to 15 carbon atoms (C₆-C₁₅ aryl). In still other embodiments, an aryl group comprises from 6 to 15 carbon atoms (C₆-C₁₀ aryl).

“Arylalkyl,” by itself or as part of another substituent, refers to an acyclic alkyl group in which one of the hydrogen atoms bonded to a carbon atom, typically a terminal or sp³ carbon atom, is replaced with an aryl group, as defined herein. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In some embodiments, an arylalkyl group is (C₆-C₃₀) arylalkyl, e.g., the alkanyl, alkenyl or alkynyl moiety of the arylalkyl group is (C₁-C₁₀) alkyl and the aryl moiety is (C₆-C₂₀) aryl. In other embodiments, an arylalkyl group is (C₆-C₂₀) arylalkyl, e.g., the alkanyl, alkenyl or alkynyl moiety of the arylalkyl group is (C₁-C₈) alkyl and the aryl moiety is (C₆-C₁₂) aryl. In still other embodiments, an arylalkyl group is (C₆-C₁₅) arylalkyl, e.g., the alkanyl, alkenyl or alkynyl moiety of the arylalkyl group is (C₁-C₅) alkyl and the aryl moiety is (C₆-C₁₀) aryl.

“Cycloalkyl,” by itself or as part of another substituent, refers to a saturated or unsaturated cyclic alkyl radical, as defined herein. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane, and the like. In some embodiments, the cycloalkyl group comprises from 3 to 10 ring atoms (C₃-C₁₀ cycloalkyl). In other embodiments, the cycloalkyl group comprises from 3 to 7 ring atoms (C₃-C₇ cycloalkyl).

“Cycloheteroalkyl,” by itself or as part of another substituent, refers to a saturated or unsaturated cyclic alkyl radical in which one or more carbon atoms (and optionally any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidone, quinuclidine, and the like. In some embodiments, the cycloheteroalkyl group comprises from 3 to 10 ring atoms (3-10 membered cycloheteroalkyl). In other embodiments, the cycloalkyl group comprise from 5 to 7 ring atoms (5-7 membered cycloheteroalkyl). A cycloheteroalkyl group may be substituted at a heteroatom, for example, a nitrogen atom, with a (C₁-C₆) alkyl group. As specific examples, N-methyl-imidazolidinyl, N-methyl-morpholinyl, N-methyl-piperazinyl, N-methyl-piperidinyl, N-methyl-pyrazolidinyl and N-methyl-pyrrolidinyl are included within the definition of “cycloheteroalkyl.” A cycloheteroalkyl group may be attached to the remainder of the molecule via a ring carbon atom or a ring heteroatom.

“Compounds” refers to compounds encompassed by structural formulae disclosed herein and includes any specific compounds within these formulae whose structure is disclosed herein. Compounds may be identified either by their chemical structure and/or chemical name. When the chemical structure and chemical name conflict, the chemical structure is determinative of the identity of the compound. The compounds described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that may be incorporated into the compounds of the invention include, but are not limited to, ²H, ³H, ¹³C, ¹⁴C, ¹⁵N, ¹⁸O, ¹⁷O, etc. Compounds may exist in unsolvated forms as well as solvated forms, including hydrated forms and as N-oxides. In general, compounds may be hydrated, solvated or N-oxides. Certain compounds may exist in multiple crystalline or amorphous forms. In general, all physical forms are equivalent for the uses contemplated herein and are intended to be within the scope of the present invention. Numerous prodrug moieties, and information concerning their selection, synthesis and use are well known in the art (see, e.g., Wuts and Green, “Protective Groups in Organic Chemistry”, (Wiley, 4^{th} ed., 2007) and Harrison et al., “Compendium of Synthetic Organic Methods”, Vols. 1-8 (John Wiley and Sons, 1971-1996). Accordingly, any prodrug forms of the compounds described herein are encompassed by the present description. Further, it should be understood, when partial structures of the compounds are illustrated, that brackets indicate the point of attachment of the partial structure to the rest of the molecule. Also it should be understood that the denotion [symbol] in compounds of Formula (I) and (II) denote a double bond which is optionally present depending on other restrictions.

“Halo,” by itself or as part of another substituent refers to a radical —F, —Cl, —Br or —I.

“Heteroalkyl,” “Heteroalkanyl,” “Heteroalkenyl” and “Heteroalkynyl,” by themselves or as part of other substituents, refer to alkyl, alkanyl, alkenyl and alkynyl groups, respectively, in which one or more of the carbon atoms (and optionally any associated hydrogen atoms), are each, independently of one another, replaced with the same or different heteroatoms or heteroatomic groups. Typical heteroatoms or heteroatomic groups which can replace the carbon atoms include, but are not limited to, —O—, —S—. —N—, —Si—, —NH—, —S(O)₂—, S(O)₂—, —S(O)NH—, —S(O)₂NH— and the like and combinations thereof. The heteroatoms or heteroatomic groups may be placed at any interior position of the alkyl, alkenyl or alkynyl groups. Typical heteroatomic groups which can be included in these groups include, but are not limited to, —O—, —S—, —O—O—, —S—S—, —O—S—, —NR⁵⁰¹R⁵⁰²—, ═N—N═, —N═N—, —N═N—NR⁵⁰³R⁴⁰⁴, —PR⁵⁰⁵—, —P(O)₂—, —POR⁵⁰⁶—, —O—P(O)₂—, —SO—, —SO₂—, —SnR⁵⁰⁷R⁵⁰⁸— and the like, where R⁵⁰¹, R⁵⁰², R⁵⁰³, R⁵⁰⁴, R⁵⁰⁵, R⁵⁰⁶, R⁵⁰⁷ and R⁵⁰⁸ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl,” by itself or as part of another substituent, refers to a monovalent heteroaromatic radical derived by the removal of one hydrogen atom from a single atom of a parent heteroaromatic ring systems, as defined herein. Typical heteroaryl groups include, but are not limited to, groups derived from acridine, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some embodiments, the heteroaryl group comprises from 5 to 20 ring atoms (5-20 membered heteroaryl). In other embodiments, the heteroaryl group comprises from 5 to 10 ring atoms (5-10 membered heteroaryl). Exemplary heteroaryl groups include those derived from furan, thiophene, pyrrole, benzothiophene, benzofuran, benzimidazole, indole, pyridine, pyrazole, quinoline, imidazole, oxazole, isoxazole and pyrazine.

“Heteroarylalkyl” by itself or as part of another substituent refers to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heteroarylalkynyl is used. In some embodiments, the heteroarylalkyl group is a 6-21 membered heteroarylalkyl, e.g., the alkanyl, alkenyl or alkynyl moiety of the heteroarylalkyl is (C₁-C₆) alkyl and the heteroaryl moiety is a 5-15-membered heteroaryl. In other embodiments, the heteroarylalkyl is a 6-13 membered heteroarylalkyl, e.g., the alkanyl, alkenyl or alkynyl moiety is (C₁-C₃) alkyl and the heteroaryl moiety is a 5-10 membered heteroaryl.

“Parent Aromatic Ring System” refers to an unsaturated cyclic or polycyclic ring system having a conjugated it electron system. Specifically included within the definition of “parent aromatic ring system” are fused ring systems in which one or more of the rings are aromatic and one or more of the rings are saturated or unsaturated, such as, for example, fluorene, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Parent Heteroaromatic Ring System” refers to a parent aromatic ring system in which one or more carbon atoms (and optionally any associated hydrogen atoms) are each independently replaced with the same or different heteroatom. Typical heteroatoms to replace the carbon atoms include, but are not limited to, N, P, O, S, Si, etc. Specifically included within the definition of “parent heteroaromatic ring system” are fused ring systems in which one or more of the rings are aromatic and one or more of the rings are saturated or unsaturated, such as, for example, benzodioxan, benzofuran, chromane, chromene, indole, indoline, xanthene, etc. Typical parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease).

“Protecting group” refers to a grouping of atoms that when attached to a reactive functional group in a molecule masks, reduces or prevents reactivity of the functional group. Examples of protecting groups can be found in Green et al., “Protective Groups in Organic Chemistry”, (Wiley, 2^{nd} ed. 1991) and Harrison et al., “Compendium of Synthetic Organic Methods”, Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

“Salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Substituted,” when used to modify a specified group or radical, means that one or more hydrogen atoms of the specified group or radical are each, independently of one another, replaced with the same or different substituent(s). Substituent groups useful for substituting saturated carbon atoms in the specified group or radical include, but are not limited to —R^{a}, halo, —O⁻, ═O, —OR^{b}, —SR^{b}, —SeR^{b}, —S⁻, ═S, —NR^{c}R^{c}, ═NR^{b}, ═N—OR^{b}, trihalomethyl, —CF₃, —CN, —OCN, —SCN, —NO, —NO₂, ═N₂, —N₃, —S(O)₂R^{b}, —S(O)₂NR^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O), —P(O)(OR^{b})(OR^{b}), —OP(O)(O⁻)₂, —OP(O)(OR^{b})(O), —OP(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a} is selected from the group consisting of alkyl, substituted alkyl, heteroalkyl, substituted heteroalkyl, aryl, substituted aryl, arylalkyl, substituted arylalkyl, heteroaryl, substituted heteroaryl, heteroarylalkyl and substituted heteroarylalkyl; each R^{b} is independently hydrogen or R^{a}; and each R^{c} is independently R^{b} or alternatively, the two R^{c}s taken together with the nitrogen atom to which they are bonded form a 4-, 5-, 6- or 7-membered cycloheteroalkyl which may optionally include from 1 to 4 of the same or different additional heteroatoms selected from the group consisting of O, N and S. As specific examples, —NR^{c}R^{c} is meant to include —NH₂, —NH-alkyl, N-pyrrolidinyl and N-morpholinyl.

Similarly, substituent groups useful for substituting unsaturated carbon atoms in the specified group or radical include, but are not limited to, —R^{a}, halo, —O⁻, —OR^{b}, —SR^{b}, —SeR^{b}, —S⁻, —NR^{c}R^{c}, trihalomethyl, —CF₃, —CN, —OCN, —SCN, —NO, —NO₂, —N₃, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c} where R^{a}, R^{b} and R^{c} are as previously defined.

Substituent groups useful for substituting nitrogen atoms in heteroalkyl and cycloheteroalkyl groups include, but are not limited to, —R^{a}, —O⁻, —OR^{b}, —SR^{b}, —S⁻, —NR^{c}R^{c}, trihalomethyl, —CF₃, —CN, —NO, —NO₂, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O), —P(O)(OR^{b})(OR^{b}), —C(O)R^{b}, —C(S)R^{b}, —C(NR^{b})R^{b}, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)OR^{b}, —OC(S)OR^{b}, —NR^{b}C(O)R^{b}, —NR^{b}C(S)R^{b}, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c} where R^{a}, R^{b} and R^{c} are as previously defined.

Substituent groups from the above lists useful for substituting other specified groups or atoms will be apparent to those of skill in the art.

The substituents used to substitute a specified group can be further substituted, typically with one or more of the same or different groups selected from the various groups specified above.

“Subject,” “individual” or “patient” are used interchangeably herein and refer to a vertebrate, preferably a mammal. Mammals include, but are not limited to, murines, rodents, simians, humans, farm animals, sport animals and pets.

“Treating” or “treatment” of any disease or disorder refers, in some embodiments, to ameliorating the disease or disorder (i.e., arresting or reducing the development of the disease or at least one of the clinical symptoms thereof). In other embodiments “treating” or “treatment” refers to ameliorating at least one physical parameter, which may not be discernible by the patient. In yet other embodiments, “treating” or “treatment” refers to inhibiting the disease or disorder, either physically, (e.g., stabilization of a discernible symptom), physiologically, (e.g., stabilization of a physical parameter) or both. In yet other embodiments, “treating” or “treatment” refers to delaying the onset of the disease or disorder.

“Therapeutically effective amount” means the amount of a compound that, when administered to a patient for treating a disease, is sufficient to effect such treatment for the disease. The “therapeutically effective amount” will vary depending on the compound, the disease and its severity and the age, weight, etc., of the patient to be treated.

“Vehicle” refers to a diluent, adjuvant, excipient or carrier with which a compound is administered.

B. Compounds

The compounds described herein are pharmaceutical agents which treat, prevent or ameliorate a variety of disorders associated with excessive formation of reactive oxygen and nitrogen species. Without being bound by theory, the compounds described herein may reduce oxidative oxygen (e.g., supeoxide, preoxides or hypochlorite) and nitrogen species (e.g., nitric oxide, nitrogendioxide or preoxynitrate) to less reactive forms. Accordingly, the compounds described herein may be used to treat, prevent or ameliorate oxidative injuries.

In some embodiments, a compound of Formula (I):

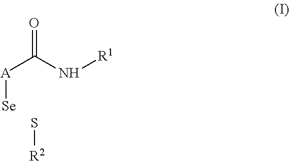

or salts, solvates or hydrates thereof is provided, wherein A is aryl, substituted aryl, heteroaryl or substituted heteroaryl; R¹ is alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl; R² is alkyl, substituted alkyl, heteroalkyl, substituted heteroalkyl, aryl, substituted aryl, arylalkyl, substituted arylalkyl, heteroaryl, substituted heteroaryl, heteroarylalkyl, substituted heteroarylalkyl,

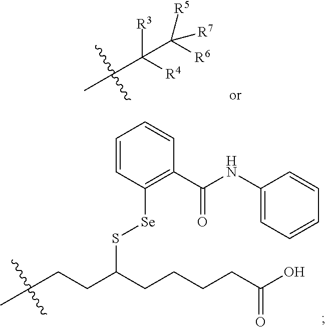

R³ is hydrogen, —OR⁹, —S(O)ₖR¹⁰, —NR¹¹R¹², —N R¹⁰COR¹³, —CO₂R¹³, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl; R⁵ is hydrogen, —OR¹⁴, —S(O)ₗR¹⁵, —NR¹⁶R¹⁷, —NR¹⁵COR¹⁸, —CO₂R¹⁸, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl; R⁷ is —CO₂R¹⁹, —CONR²⁰R²¹, —NR²²R²³, —NR²⁴COR¹⁹ or —S(O)ₘR²⁴; R⁴, R⁶ and R⁹-R²⁴ are independently hydrogen, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl; and k, l and m are independently 0, 1 or 2; provided that when A and R¹ are phenyl substituted with natural amounts of deuterium that R² is not

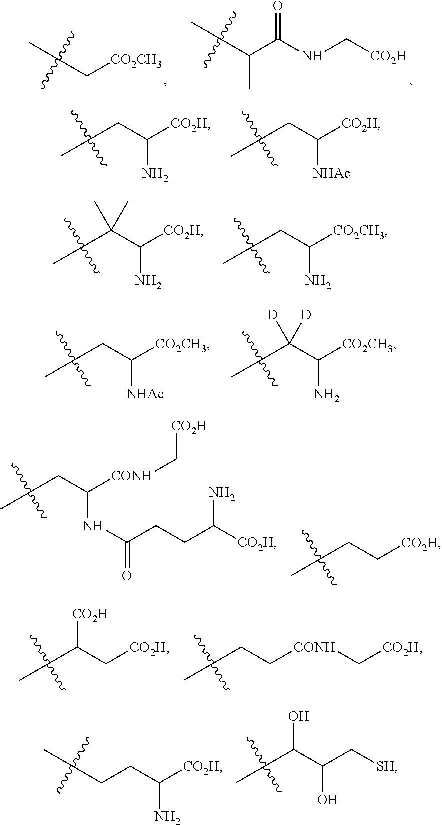

ethyl, allyl, n-propyl, isopropyl, t-butyl, n-butyl, n-hepyl, n-octyl, cyclohexyl, 4-trans-t-butylcyclohexyl, 4-cis-t-butylcyclohexyl, phenyl, p-methylphenyl, p-chlorophenyl, o-benzoic acid, o-methyl benzoate, 2-napthyl, benzyl, 2-pyridyl,

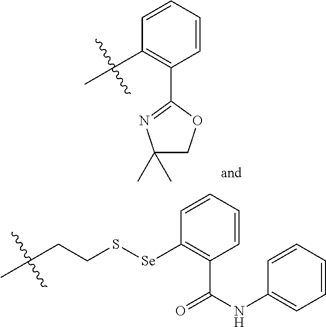

wherein the above compounds are substituted with natural amounts of deuterium, except where deuterium is explicitly specified in the structure of the above compounds.

In some embodiments, A is aryl, substituted aryl, phenyl or substituted phenyl. In other embodiments, A is phenyl.

In some embodiments, R² is substituted alkyl, heteroalkyl, substituted heteroalkyl, or

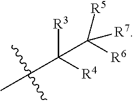

In other embodiments, R² is substituted alkyl or

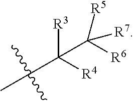

In still other embodiments, R³ is hydrogen, —OR⁹, —CO₂R¹³, alkyl or substituted alkyl. In still other embodiments, R⁵ is hydrogen, —OR¹⁴, alkyl or substituted alkyl. In still other embodiments, R⁷ is —CO₂R¹⁹ or —CONR²⁰R²¹.

In some embodiments, R⁴, R⁶ and R⁹-R²⁴ are independently hydrogen, alkyl, substituted alkyl, aryl, substituted aryl, heteroaryl or substituted heteroaryl. In other embodiments, R⁴, R⁶ and R⁹-R²⁴ are independently hydrogen, alkyl or substituted alkyl.

In some embodiments, R¹ is aryl, substituted aryl, heteroaryl or substituted heteroaryl. In other embodiments, R¹ is aryl, substituted aryl, phenyl or substituted phenyl. In still other embodiments, R¹ is phenyl or substituted phenyl. In still other embodiments, R¹ is phenyl.

In some embodiments, A is aryl, substituted aryl, phenyl or substituted phenyl wherein R¹ is aryl, substituted aryl, phenyl or substituted phenyl R² is substituted alkyl, heteroalkyl, substituted heteroalkyl, or

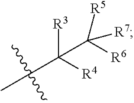

R³ is hydrogen, —OR⁹, —CO₂R¹³, alkyl or substituted alkyl; R⁵ is hydrogen, —OR¹⁴, alkyl or substituted alkyl; and R⁴, R⁶ and R⁹-R²⁴ are independently hydrogen, alkyl, substituted alkyl, aryl, substituted aryl, heteroaryl or substituted heteroaryl.

In some embodiments, A is phenyl; R¹ is phenyl; R² is substituted alkyl, or

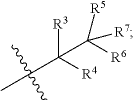

R³ is hydrogen, —OR⁹, —CO₂R¹³, alkyl or substituted alkyl; R⁵ is hydrogen, —OR¹⁴, alkyl or substituted alkyl; and R⁴, R⁶ and R⁹-R²⁴ are independently hydrogen, alkyl or substituted alkyl.

In some embodiments, R² is

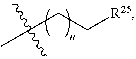

R²⁵ is —OR²⁶, —S(O)ⱼR²⁷, —NR²⁸R²⁹, —CONR²⁸R²⁹, —CO₂R³⁰, —PO(OR³¹)(OR³²), —OPO(OR³¹)(OR³²) or —NCNHR³³(NHR³⁴R³⁵), R²⁶-R³⁴ are independently hydrogen, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl; is an integer from 0 to 9; and j is 1, 2 or 3. In other embodiments, R² is

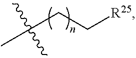

R²⁵ is —OR²⁶, —S(O)ⱼR²⁷, —NR²⁸R²⁹, —CONR²⁸R²⁹, —CO₂R³⁰, —PO(OR³¹)(OR³²), —OPO(OR³¹)(OR³²) or —NCNHR³³(NHR³⁴R³⁵) and R²⁶-R²⁹ and R³¹-R³⁴ are hydrogen, R³⁰ is hydrogen, methyl or ethyl; n is an integer from 0 to 9; and j is 1, 2 or 3. In still other embodiments, R² is

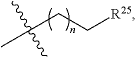

R²⁵ is —OR²⁶, —S(O)ⱼR²⁷, —NR²⁸R²⁹, —CONR²⁸R²⁹, —CO₂R³⁰, —PO(OR³¹)(OR³²), —OPO(OR³¹)(OR³²) or —NCNHR³³(NHR³⁴R³⁵) and R²⁶-R²⁹ and R³¹-R³⁴ are hydrogen, R³⁰ is hydrogen, methyl or ethyl; n is 1; and j is 1, 2 or 3. In still other embodiments, R² is

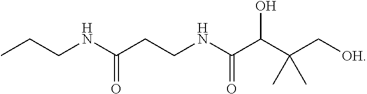

In still other other embodiments, R² is

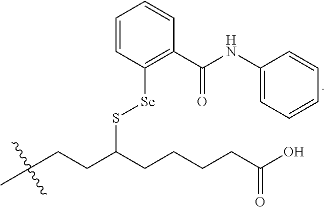

In some of the preceding embodiments in this paragraph, A is aryl, substituted aryl, phenyl or substituted phenyl. In other of the preceding embodiments in this paragraph, A is phenyl. In still other of the preceding embodiments in this paragraph, R² is aryl, substituted aryl, phenyl or substituted phenyl. In still other of the preceding embodiments in this paragraph, R² is phenyl. In still other of the preceding embodiments in this paragraph, A is aryl, substituted aryl, phenyl or substituted phenyl and R² is aryl, substituted aryl, phenyl or substituted phenyl. In still other of the preceding embodiments in this paragraph, A is phenyl and R² is phenyl.

In some embodiments, R³ and R⁴ are independently hydrogen or alkyl. In other embodiments, R³ and R⁴ are independently hydrogen or methyl. In still other embodiments, R³ and R⁴ are hydrogen or R³ and R⁴ are methyl. In still other embodiments, R⁵ is hydrogen and R⁶ is alkyl, —NR¹⁶R¹⁷ or —NR¹⁵COR¹⁸. In still other embodiments, R⁵ is hydrogen and R⁶ is alkyl, —NH₂ or —NHCOR¹⁸. In still other embodiments, R⁵ is hydrogen and R⁶ is —CH₃, —COCH₃, —CH₂CH₂CH(NH₂)CO₂H, —CH₂CH₂CH(NHCOCH₃)CO₂H, or —CH₂CH₂CH(NH₂)COCH₂CH₃.

In still other embodiments, R⁷ is —CO₂R¹⁹ or —CONR²⁰R²¹. In still other embodiments, R⁷ is —CO₂R¹⁹ or —CONR²⁰R²¹, R¹⁹ is hydrogen or alkyl and R²⁰ and R²¹ are hydrogen, alkyl or substituted alkyl. In still other embodiments, R⁷ is —CO₂H, —CO₂CH₂CH₃, —CONHCH₂CO₂H, —CONHCH₂CO₂CH₂CH₃ or

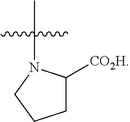

In some the preceding embodiments in this paragraph, A is aryl, substituted aryl, phenyl or substituted phenyl and R² is aryl, substituted aryl, phenyl or substituted phenyl. In other the preceding embodiments in this paragraph, A is phenyl and R² is phenyl.

In some embodiment, R³ and R⁴ are independently hydrogen or alkyl, R⁵ is hydrogen, R⁶ is alkyl, —NR¹⁶R¹⁷ or —NR¹⁵COR¹⁸ and R⁷ is —CO₂R¹⁹ or —CONR²⁰R²¹. In other embodiments, R³ and R⁴ are independently hydrogen or methyl, R⁵ is hydrogen, R⁶ is alkyl, —NH₂ or —NHCOR¹⁸, R⁷ is —CO₂R¹⁹ or —CONR²⁰R²¹, R¹⁹ is hydrogen or alkyl and R²⁰ and R²¹ are hydrogen, alkyl or substituted alkyl. In still other embodiments, R³ and R⁴ are hydrogen or R³ and R⁴ are methyl, R⁵ is hydrogen, R⁶ is —CH₃, —COCH₃, —CH₂CH₂CH(NH₂)CO₂H, —CH₂CH₂CH(NHCOCH₃)CO₂H, —CH₂CH₂CH(NH₂)COCH₂CH₃ and R⁷ is —CO₂H, —CO₂CH₂CH₃, —CONHCH₂CO₂H, —CONHCH₂CO₂CH₂CH₃ or

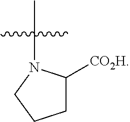

In some of the preceding embodiments in this paragraph, A is aryl, substituted aryl, phenyl or substituted phenyl and R² is aryl, substituted aryl, phenyl or substituted phenyl. In other of the preceding embodiments in this paragraph, A is phenyl and R² is phenyl.

In some embodiments, the compounds of Formula (I) have the structure:

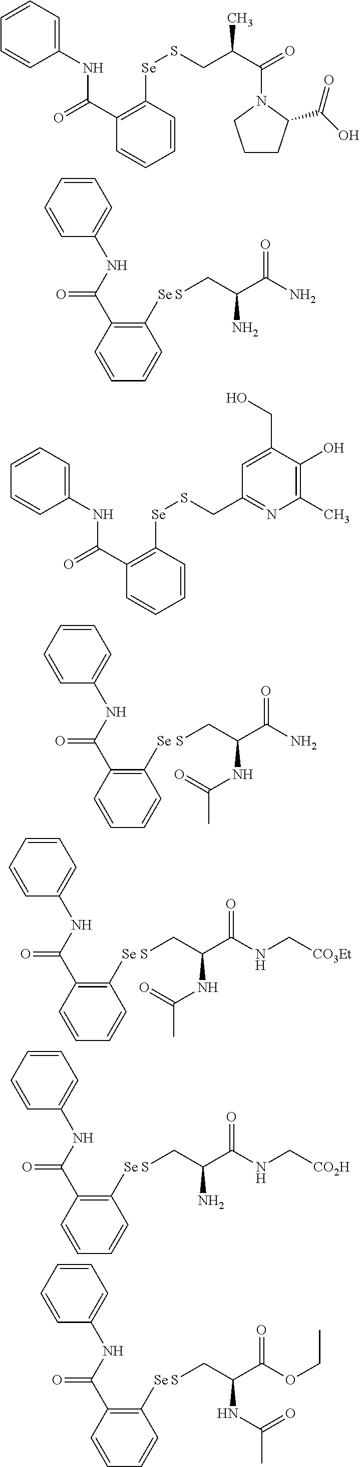

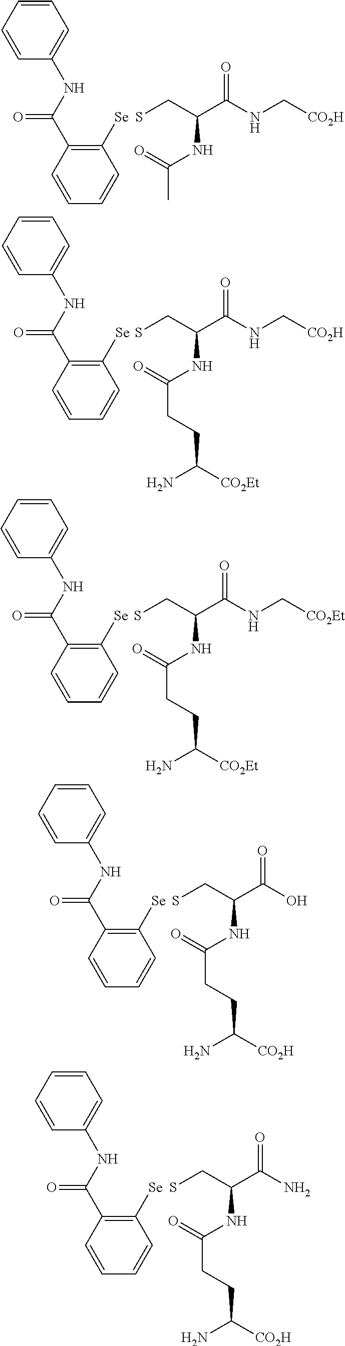

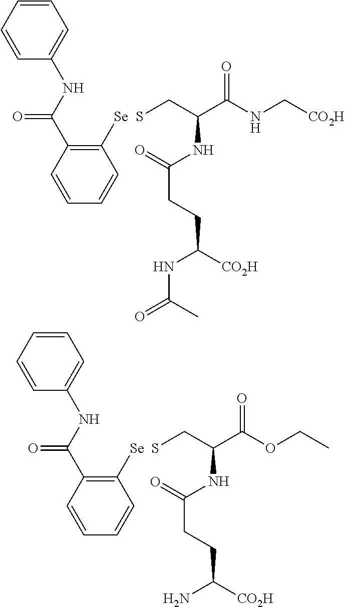

In some embodiments, a compound of Formula (II):

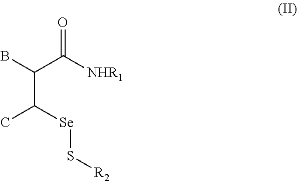

or salts, solvates or hydrates thereof is provided, wherein B and C together with the atoms to which they are bonded form an aryl, substituted aryl, heteroaryl or substituted heteroaryl ring; R¹ is alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl; R² is alkyl, substituted alkyl, heteroalkyl, substituted heteroalkyl, aryl, substituted aryl, arylalkyl, substituted arylalkyl, heteroaryl, substituted heteroaryl, heteroarylalkyl, substituted heteroarylalkyl,

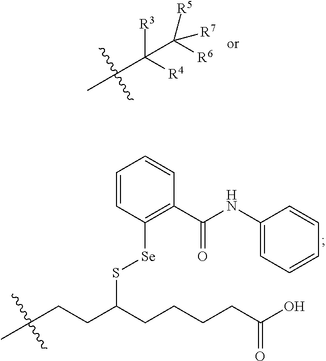

R³ is hydrogen, —OR⁹, —S(O)ₖR¹⁰, —NR¹¹R¹², —NR¹⁰COR¹³, —CO₂R¹³, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl; R⁵ is hydrogen, —OR¹⁴, —S(O)ₗR¹⁵, —NR¹⁶R¹⁷, —N R¹⁵COR¹⁸, —CO₂R¹⁸, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl; R⁷ is —CO₂R¹⁹, —CONR²⁰R²¹, —NR²²R²³, —NR²⁴COR¹⁹ or —S(O)ₘR²⁴; R⁴, R⁶ and R⁹-R²⁴ are independently hydrogen, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl; and k, l and m are independently 0, 1 or 2; provided that when A and R¹ are phenyl substituted with natural amounts of deuterium that R² is not

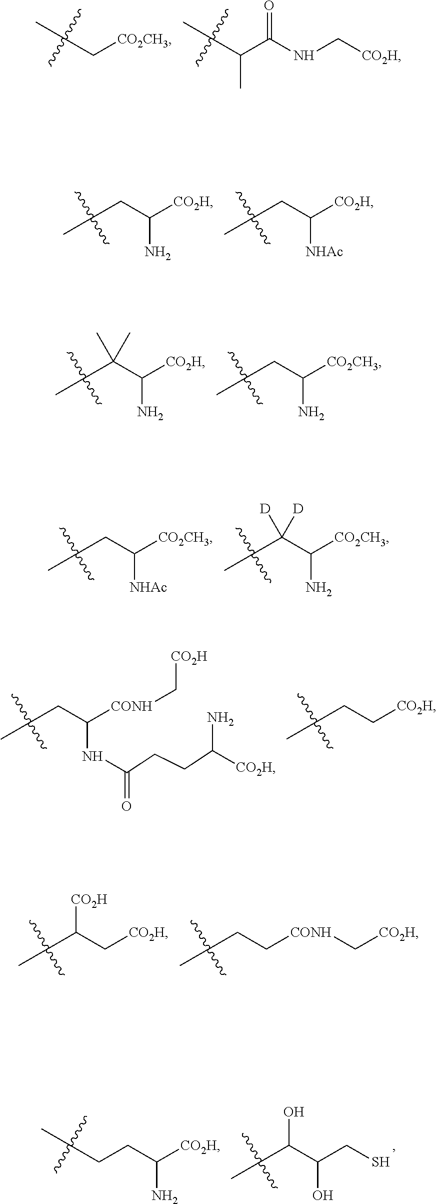

ethyl, allyl, n-propyl, isopropyl, t-butyl, n-butyl, n-hepyl, n-octyl, cyclohexyl, 4-trans-t-butylcyclohexyl, 4-cis-t-butylcyclohexyl, phenyl, p-methylphenyl, p-chlorophenyl, o-benzoic acid, o-methyl benzoate, 2-napthyl, benzyl, 2-pyridyl,

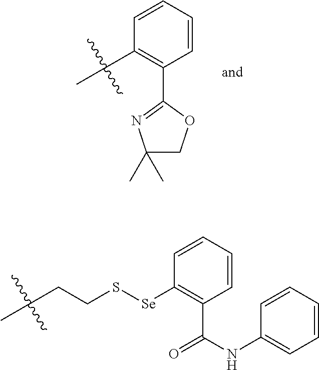

wherein the above compounds are substituted with natural amounts of deuterium, except where deuterium is explicitly specified in the structure of the above compounds.

In some embodiments, R² is substituted alkyl, heteroalkyl, substituted heteroalkyl, or

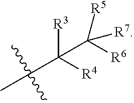

In other embodiments, R² is substituted alkyl

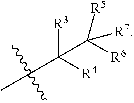

In still other embodiments, R³ is hydrogen, —OR⁹, —CO₂R¹³, alkyl or substituted alkyl. In other R⁵ is hydrogen, —OR¹⁴, alkyl or substituted alkyl. In still other embodiments, R⁷ is —CO₂R¹⁹ or —CONR²⁰R²¹.

In some embodiments, R⁴, R⁶ and R⁹-R²⁴ are independently hydrogen, alkyl, substituted alkyl, aryl, substituted aryl, heteroaryl or substituted heteroaryl.

In other embodiments, R⁴, R⁶ and R⁹-R²⁴ are independently hydrogen, alkyl or substituted alkyl.

In some embodiments, R¹ is aryl, substituted aryl, heteroaryl or substituted heteroaryl. In other embodiments, R¹ is aryl, substituted aryl, phenyl or substituted phenyl. In still other embodiments, R¹ is phenyl or substituted phenyl. In still other embodiments, R¹ is phenyl.

In some embodiments, B and C form an aryl, substituted aryl, phenyl or substituted phenyl ring; R¹ is aryl, substituted aryl, phenyl or substituted phenyl; R² is substituted alkyl, heteroalkyl, substituted heteroalkyl, or

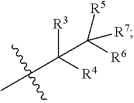

R³ is hydrogen, —OR⁹, —CO₂R¹³, alkyl or substituted alkyl; R⁵ is hydrogen-OR¹⁴, alkyl or substituted alkyl; and R⁴, R⁶ and R⁹-R²⁴ are independently hydrogen, alkyl, substituted alkyl, aryl, substituted aryl, heteroaryl or substituted heteroaryl. In other embodiments, B and C form a phenyl ring; R¹ is phenyl; R² is substituted alkyl, or

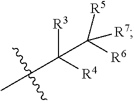

R³ is hydrogen, —OR⁹, —CO₂R¹³, alkyl or substituted alkyl; R⁵ is hydrogen, —OR¹⁴, alkyl or substituted alkyl; and R⁴, R⁶ and R⁹-R²⁴ are independently hydrogen, alkyl or substituted alkyl.

In some embodiments, R² is

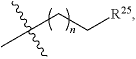

R²⁵ is, —OR²⁶, —S(O)ⱼR²⁷, —NR²⁸R²⁹, —CONR²⁸R²⁹, —CO₂R³⁰, —PO(OR³¹)(OR³²), —OPO(OR³¹)(OR³²) or —NCNHR³³(NHR³⁴R³⁵) and R²⁶-R³⁴ are independently hydrogen, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl; n is an integer from 0 to 9; and j is 1, 2 or 3. In other embodiments, R² is

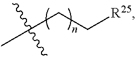

R²⁵ is, —OR²⁶, —S(O)ⱼR²⁷, —NR²⁸R²⁹, —CONR²⁸R²⁹, —CO₂R³⁰, —PO(OR³¹)(OR³²), —OPO(OR³¹)(OR³²) or —NCNHR³³(NHR³⁴R³⁵) and R²⁶-R²⁹ and R³¹-R³⁴ are hydrogen and R³⁰ is hydrogen, methyl or ethyl; n is an integer from 0 to 9; and j is 1, 2 or 3. In still other embodiments, R² is

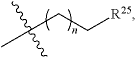

R²⁵ is, —OR²⁶, —S(O)ⱼR²⁷, —NR²⁸R²⁹, —CONR²⁸R²⁹, —CO₂R³⁰, —PO(OR³¹)(OR³²), —OPO(OR³¹)(OR³²) or —NCNHR³³(NHR³⁴R³⁵) and R²⁶-R²⁹ and R³¹-R³⁴ are hydrogen and R³⁰ is hydrogen, methyl or ethyl; n is 1; and j is 1, 2 or 3. In still other embodiments, R² is

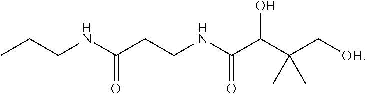

In still other embodiments, R² is

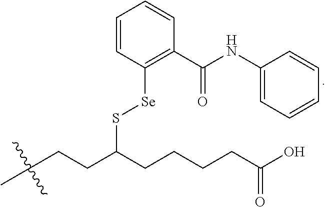

In some of the preceding embodiments in this paragraph, B and C form a phenyl ring. In other of the preceding embodiments in this paragraph, R² is aryl, substituted aryl, phenyl or substituted phenyl. In still other of the preceding embodiments in this paragraph, R² is phenyl. In still other of the preceding embodiments in this paragraph, B and C form a phenyl ring and R² is aryl, substituted aryl, phenyl or substituted phenyl. In still other of the preceding embodiments in this paragraph, B and C form a phenyl ring and R² is phenyl.

In some embodiments, R³ and R⁴ are independently hydrogen or alkyl. In other embodiments, R³ and R⁴ are independently hydrogen or methyl. In still other embodiments, R³ and R⁴ are hydrogen or R³ and R⁴ are methyl.

In some embodiments, R⁵ is hydrogen and R⁶ is alkyl, —NR¹⁶R¹⁷ or —NR¹⁵COR¹⁸. In other embodiments, R⁵ is hydrogen and R⁶ is alkyl, —NH₂ or —NHCOR¹⁸. In still other embodiments, R⁵ is hydrogen and R⁶ is —CH₃, —COCH₃, —CH₂CH₂CH(NH₂)CO₂H, —CH₂CH₂CH(NHCOCH₃)CO₂H or —CH₂CH₂CH(NH₂)COCH₂CH₃. In some embodiments, R⁷ is —CO₂R¹⁹ or —CONR²⁰R²¹. In other embodiments, R⁷ is —CO₂R¹⁹ or —CONR²⁰R²¹ and R¹⁹ is hydrogen or alkyl and R²⁰ and R²¹ are hydrogen, alkyl or substituted alkyl. In still other embodiments, R⁷ is —CO₂H, —CO₂CH₂CH₃, —CONHCH₂CO₂H, —CONHCH₂CO₂CH₂CH₃ or

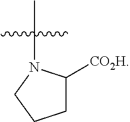

In some of the preceding embodiments in this paragraph, B and C form a phenyl ring and R² is aryl, substituted aryl, phenyl or substituted phenyl. In other of the preceding embodiments in this paragraph, B and C form a phenyl ring and R² is phenyl.

In some embodiments, R³ and R⁴ are independently hydrogen or alkyl, R⁵ is hydrogen and R⁶ is alkyl, —NR¹⁶R¹⁷ or —NR¹⁵COR¹⁸ and R⁷ is —CO₂R¹⁹ or —CONR²⁰R²¹. In other embodiments, R³ and R⁴ are independently hydrogen or methyl, R⁵ is hydrogen and R⁶ is alkyl, —NH₂ or —NHCOR¹⁸ and R⁷ is —CO₂R¹⁹ or —CONR²⁰R²¹ and R¹⁹ is hydrogen or alkyl and R²⁰ and R²¹ are hydrogen, alkyl or substituted alkyl. In still other embodiments, R³ and R⁴ are hydrogen or R³ and R⁴ are methyl, R⁵ is hydrogen and R⁶ is —CH₃, —COCH₃, —CH₂CH₂CH(NH₂)CO₂H, —CH₂CH₂CH(NHCOCH₃)CO₂H, —CH₂CH₂CH(NH₂)COCH₂CH₃. and R⁷ is —CO₂H, —CO₂CH₂CH₃, —CONHCH₂CO₂H, —CONHCH₂CO₂CH₂CH₃ or

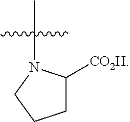

In some of the preceding embodiments in this paragraph, B and C form a phenyl ring and R² is aryl, substituted aryl, phenyl or substituted phenyl. In other of the preceding embodiments in this paragraph, B and C form a phenyl ring and R² is phenyl.

In some embodiments, a compound of Formula (III):

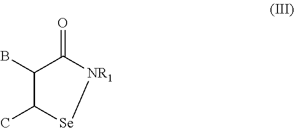

or salts, solvates or hydrates thereof is provided, wherein:
B and C together with the atoms to which they are bonded form an aryl, substituted aryl, heteroaryl or substituted heteroaryl ring; and R¹ is alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl wherein at least one hydrogen in Formula (III) is substituted with deuterium.

In some embodiments, R¹ is aryl, substituted aryl, phenyl or substituted phenyl. In other embodiments, R¹ is phenyl or substituted phenyl. In still other embodiments, R¹ is phenyl. In some of the preceding embodiments of this paragraph, B and C form a phenyl ring.

In some embodiments, the compounds of Formula (III) have the structure:

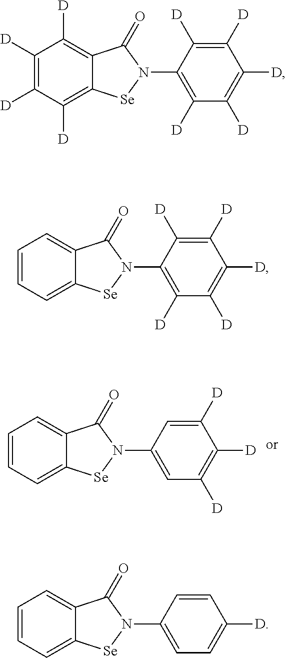

The compounds described herein may be prepared by method known to those of skill in the art such as those described in various compendia such as, for example; Harrison et al., “Compendium of Synthetic Organic Methods”, Vols. 1-8 (John Wiley and Sons, 1971-1996); “Beilstein Handbook of Organic Chemistry,” Beilstein Institute of Organic Chemistry, Frankfurt, Germany; Feiser et al., “Reagents for Organic Synthesis,” Volumes 1-17, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” John Wiley & Sons, 1995, and various journal articles including, but not limited to, Yurugi et al., Yakugaku Zasshi 1960 80 1686-91, Lesser et al., Ber. Dtsch. Chem. Ges. 1924 57 1077, Engman et al., J. Org. Chem. 1989 54 2964-66, Haenen et al., Molecular Pharmacology 1990 37(3) 412-422. and Glass et al., J. Org. Chem., 1989 54 1092-1097.

C. Compositions and Methods of Administration

The compositions provided herein contain therapeutically effective amounts of one or more of the compounds provided herein that are useful in the prevention, treatment, or amelioration of one or more of the symptoms of diseases or disorders described herein and a vehicle. Vehicles suitable for administration of the compounds provided herein include any such carriers known to those skilled in the art to be suitable for the particular mode of administration.

In addition, the compounds may be formulated as the sole active ingredient in the composition or may be combined with other active ingredients.

The compositions contain one or more compounds provided herein. The compounds are, in some embodiments, formulated into suitable preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In some embodiments, the compounds described above are formulated into compositions using techniques and procedures well known in the art (see, e.g., Ansel Introduction to Pharmaceutical Dosage Forms, Seventh Edition (1999).

In the compositions, effective concentrations of one or more compounds or derivatives thereof is (are) mixed with a suitable vehicle. The compounds may be derivatized as the corresponding salts, esters, enol ethers or esters, acetals, ketals, orthoesters, hemiacetals, hemiketals, acids, bases, solvates, hydrates or prodrugs prior to formulation, as described above. The concentrations of the compounds in the compositions are effective for delivery of an amount, upon administration, that treats, prevents, or ameliorates one or more of the symptoms of diseases or disorders described herein. In some embodiments, the compositions are formulated for single dosage administration. To formulate a composition, the weight fraction of a compound is dissolved, suspended, dispersed or otherwise mixed in a selected vehicle at an effective concentration such that the treated condition is relieved, prevented, or one or more symptoms are ameliorated.

The active compound is included in the vehicle in an amount sufficient to exert a therapeutically useful effect in the absence of undesirable side effects on the patient treated. The therapeutically effective concentration may be determined empirically by testing the compounds in in vitro and in vivo systems well known to those of skill in the art and then extrapolated therefrom for dosages for humans.

The concentration of active compound in the composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of diseases or disorders described herein.

In some embodiments, a therapeutically effective dosage should produce a serum concentration of active ingredient of from about 0.1 ng/ml to about 50-100 μg/ml. The compositions, in other embodiments, should provide a dosage of from about 0.001 mg to about 2000 mg of compound per kilogram of body weight per day. Dosage unit forms are prepared to provide from about 0.01 mg, 0.1 mg or 1 mg to about 500 mg, 1000 mg or 2000 mg, and in some embodiments from about 10 mg to about 500 mg of the active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions and that the concentration and ranges set forth herein are exemplary only and are not intended to limit the scope or practice of the described compositions.

In instances in which the compounds exhibit insufficient solubility, methods for solubilizing compounds may be used. Such methods are known to those of skill in this art, and include, but are not limited to, using co-solvents, such as dimethylsulfoxide (DMSO), using surfactants, such as TWEEN®, or dissolution in aqueous sodium bicarbonate. Derivatives of the compounds, such as prodrugs of the compounds may also be used in formulating effective compositions.

Upon mixing or addition of the compound(s), the resulting mixture may be a solution, suspension, emulsion or the like. The form of the resulting mixture depends upon a number of factors, including the intended mode of administration and the solubility of the compound in the selected vehicle. The effective concentration is sufficient for ameliorating the symptoms of the disease, disorder or condition treated and may be empirically determined.

The compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The therapeutically active compounds and derivatives thereof are, in some embodiments, formulated and administered in unit-dosage forms or multiple-dosage forms. Unit-dose forms as used herein refer to physically discrete units suitable for human and animal subjects and packaged individually as is known in the art. Each unit-dose contains a predetermined quantity of the therapeutically active compound sufficient to produce the desired therapeutic effect, in association with the required vehicle. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid administrable compositions can, for example, be prepared by dissolving, dispersing, or otherwise mixing an active compound as defined above and optional adjuvants in a vehicle, such as, for example, water, saline, aqueous dextrose, glycerol, glycols, ethanol, and the like, to thereby form a solution or suspension. If desired, the composition to be administered may also contain minor amounts of nontoxic auxiliary substances such as wetting agents, emulsifying agents, solubilizing agents, pH buffering agents and the like, for example, acetate, sodium citrate, cyclodextrin derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975 or later editions thereof.

Dosage forms or compositions containing active ingredient in the range of 0.005% to 100% with the balance made up from non-toxic carrier may be prepared. Methods for preparation of these compositions are known to those skilled in the art. The contemplated compositions may contain 0.001%-100% active ingredient, in one embodiment 0.1-95%, in another embodiment 75-85%.

In certain embodiments, the compositions are lactose-free compositions containing excipients that are well known in the art and are listed, for example, in the U.S. Pharmacopeia (USP) 25-NF20 (2002). In general, lactose-free compositions contains active ingredients, a binder/filler, and a lubricant in pharmaceutically compatible and pharmaceutically acceptable amounts. Particular lactose-free dosage forms contain active ingredients, microcrystalline cellulose, pre-gelatinized starch, and magnesium stearate.

Further provided are anhydrous compositions and dosage forms comprising active ingredients, since water can facilitate the degradation of some compounds. For example, the addition of water (e.g., 5%) is widely accepted in the art as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous compositions and dosage forms provided herein can be prepared using anhydrous or low moisture containing ingredients and low moisture or low humidity conditions.

An anhydrous composition should be prepared and stored such that its anhydrous nature is maintained. Accordingly, anhydrous compositions are generally packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Oral dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms such as for example, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polyinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The compound, or derivative thereof, can be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or derivative thereof as described herein. Higher concentrations, up to about 98% by weight of the active ingredient may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Vehicles used in elixirs include solvents. Syrups are concentrated aqueous solutions of a sugar, for example, sucrose, and may contain a preservative. An emulsion is a two-phase system in which one liquid is dispersed in the form of small globules throughout another liquid. Vehicles used in emulsions are non-aqueous liquids, emulsifying agents and preservatives. Suspensions use suspending agents and preservatives. Substances used in non-effervescent granules, to be reconstituted into a liquid oral dosage form, include diluents, sweeteners and wetting agents. Substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example, propylene carbonate, vegetable oils or triglycerides, is in some embodiments encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid vehicle, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or poly-alkylene glycol, including, but not limited to, 1,2-dimethoxyethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

Parenteral administration, in some embodiments characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Vehicles used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose and polyvinylpyrrolidone. Emulsifying agents include Polysorbate 80 (TWEEN® 80). A sequestering or chelating agent of metal ions include EDTA. Vehicles also include ethyl alcohol, polyethylene glycol and propylene glycol for water miscible vehicles; and sodium hydroxide, hydrochloric acid, citric acid or lactic acid for pH adjustment.

The concentration of active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; 6,699,500 and 6,740,634. Such dosage forms can be used to provide slow or controlled-release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In some embodiments, a pump may be used (see, Sefton, CRC Crit. Ref Biomed. Eng. 14:201 (1987); Buchwald et al., Surgery 88:507 (1980); Saudek et al., N. Engl. J. Med. 321:574 (1989). In other embodiments, polymeric materials can be used. In still other embodiments, a controlled release system can be placed in proximity of the therapeutic target, i.e., thus requiring only a fraction of the systemic dose (see, e.g., Goodson, Medical Applications of Controlled Release, vol. 2, pp. 115-138 (1984). In some embodiments, a controlled release device is introduced into a subject in proximity of the site of inappropriate immune activation or a tumor. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a derivative thereof, in a suitable solvent. The solvent may contain an excipient which improves the stability or other pharmacological component of the powder or reconstituted solution, prepared from the powder. Excipients that may be used include, but are not limited to, an antioxidant, a buffer and a bulking agent. In some embodiments, the excipient is selected from dextrose, sorbital, fructose, corn syrup, xylitol, glycerin, glucose, sucrose and other suitable agent. The solvent may contain a buffer, such as citrate, sodium or potassium phosphate or other such buffer known to those of skill in the art at, at about neutral pH. Subsequent sterile filtration of the solution followed by lyophilization under standard conditions known to those of skill in the art provides the desired formulation. In some embodiments, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, in some embodiments, have diameters of less than 50 microns, in other embodiments less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

For nasal administration, the preparation may contain an esterified phosphonate compound dissolved or suspended in a liquid carrier, in particular, an aqueous carrier, for aerosol application. The carrier may contain solubilizing agents such as propylene glycol, surfactants, absorption enhancers such as lecithin or cyclodextrin, or preservatives.

These solutions, particularly those intended for ophthalmic use, may be formulated as 0.01%-10% isotonic solutions, pH about 5-7, with appropriate salts.

Other routes of administration, such as transdermal patches, including iontophoretic and electrophoretic devices, and rectal administration, are also contemplated herein.

Transdermal patches, including iotophoretic and electrophoretic devices, are well known to those of skill in the art. For example, such patches are disclosed in U.S. Pat. Nos. 6,267,983, 6,261,595, 6,256,533, 6,167,301, 6,024,975, 6,010,715, 5,985,317, 5,983,134, 5,948,433, and 5,860,957.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in one embodiment, is about 2 to 3 gm. Tablets and capsules for rectal administration are manufactured using the same pharmaceutically acceptable substance and by the same methods as for formulations for oral administration.

The compounds provided herein, or derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In some embodiments, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The compounds or derivatives may be packaged as articles of manufacture containing packaging material, a compound or pharmaceutically acceptable derivative thereof provided herein, which is effective for treatment, prevention or amelioration of one or more symptoms of diseases or disorders described herein, within the packaging material, and a label that indicates that the compound or composition, or derivative thereof, is used for the treatment, prevention or amelioration of one or more symptoms of diseases or disorders described herein.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease or disorder associated with viral infections or inappropriate cell proliferation.

In human therapeutics, the physician will determine the dosage regimen that is most appropriate according to a preventive or curative treatment and according to the age, weight, stage of the disease and other factors specific to the subject to be treated. The compositions, in other embodiments, should provide a dosage of from about 0.001 mg to about 2000 mg of compound per kilogram of body weight per day. Dosage unit forms are prepared, e.g., to provide from about 0.01 mg, 0.1 mg or 1 mg to about 500 mg, 1000 mg or 2000 mg, and in some embodiments from about 10 mg to about 500 mg of the active ingredient or a combination of essential ingredients per dosage unit form.

The amount of active ingredient in the formulations provided herein, which will be effective in the prevention or treatment of a disorder or one or more symptoms thereof, will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject.

Exemplary doses of a formulation include milligram or microgram amounts of the active compound per kilogram of subject or sample weight (e.g., from about 1 micrograms per kilogram to about 50 milligrams per kilogram, from about 10 micrograms per kilogram to about 30 milligrams per kilogram, from about 100 micrograms per kilogram to about 10 milligrams per kilogram, or from about 100 microgram per kilogram to about 5 milligrams per kilogram).

It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the composition provided herein are also encompassed by the above described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, administration of the same formulation provided herein may be repeated and the administrations may be separated by at least 1 day, 2 days, 3 days, 5 days, 10 days, 15 days, 30 days, 45 days, 2 months, 75 days, 3 months, or 6 months.

D. Methods of Use of the Compounds and Compositions

Methods of treating, preventing, or ameliorating one or more symptoms of diseases described, infra, using the compounds and compositions are provided. In practicing the methods, effective amounts of the compounds or compositions containing therapeutically effective concentrations of the compounds are administered. In certain embodiments, the methods provided herein are for the preventing, or ameliorating one or more symptoms of Absence of the Septum Pellucidum, Acid Lipase Disease, Acquired Epileptiform Aphasia, Acute Disseminated Encephalomyelitis, ADHD, Adie's Pupil, Adie's Syndrome, Adrenoleukodystrophy, Agenesis of the Corpus Callosum, Agnosia, Aicardi Syndrome, AIDS-Neurological Complications, Alexander Disease, Alpers' Disease, Alternating Hemiplegia, Alzheimer's Disease, Amyotrophic Lateral Sclerosis, Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arterio-venous Malformation, Asperger Syndrome, Ataxia, Ataxia Telangiectasia, Ataxias and Cerebellar/Spinocerebellar Degeneration, Attention Deficit-Hyperactivity Disorder, Autism, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysm, Cerebral Arteriosclerosis, Cerebral Atrophy, Cerebral Beriberi, Cerebral Gigantism, Cerebral Hypoxia, Cerebral Palsy, Cerebro-Oculo-Facio-Skeletal Syndrome, Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, COFS, Colpocephaly, Coma and Persistent Vegetative State, Complex Regional Pain Syndrome, Congenital Facial Diple-gia, Congenital Myasthenia, Congenital Myopathy, Congenital Vascular Cavernous Malformations, Corticobasal Degeneration, Cranial Arteritis, Craniosynostosis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Deep Brain Stimulation for Parkinson's Disease, Dejerine-Klumpke Palsy, Dementia, Dementia-Multi-Infarct, Dementia—Semantic, Dementia-Subcorti-cal, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Dysautonomia, Dys-graphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalotrigeminal Angiomatosis, Epilepsy, Erb-Duchenne and Dejerine-Klumpke Palsies, Erb's Palsy, Essential Tremor, Extrapon-tine Myelinolysis, Fabry's Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fisher Syndrome, Floppy Infant Syndrome, Friedreich's Ataxia, Frontotemporal Dementia, Gangliosidoses, Gaucher's Disease, Gerstmann's Syndrome, Gerstmann-Straussler-Scheinker Disease, Giant Cell Arteritis, Giant Cell Inclusion Disease, Globoid Cell Leu-kodystrophy, Glossopharyngeal Neuralgia, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neu-roaxonal Dystrophy, Infantile Phytanic Acid Storage Disease, Infantile Refsum Disease, Infantile Spasms, Inflammatory Myopathy, Iniencephaly, Intestinal Lipodystrophy, Intracranial Cysts, Intracranial Hypertension, Isaac's Syndrome, Kearns-Sayre Syndrome, Kennedy's Disease, Kins-bourne syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kliiver-Bucy Syndrome, Korsakoffs Amnesic Syndrome, Krabbe Disease, Kugelberg-Welander Disease, Kuru, Lambert-Eaton Myasthenic Syndrome, Landau-Kleffer Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leu-kodystrophy, Levine-Critchley Syndrome, Lewy Body Dementia, Lipid Storage Diseases, Lissencephaly, Locked-In Syndrome, Lou Gehrig's Disease, Lupus—Neurological Sequelae, Lyme Disease—Neurological Complications, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini-Strokes, Mitochondrial Myopathies, Mobius Syndrome, Monomelic Amyotrophy, Motor Neuron Diseases, Moyamoya Disease, Mucolipidoses, Mucopolysaccharidoses, Multifocal Motor Neuropathy, Multi-Infarct Dementia, Multiple Sclerosis, Multiple System Atrophy, Multiple System Atrophy with Orthostatic Hypotension, Muscular Dystrophy, Myasthenia-Congenital, Myasthenia Gravis, Myelinoclastic Diffuse Sclerosis, Myoclonic Encephalopathy of Infants, Myoclonus, Myopathy, Myopathy-Congenital, Myopathy-Thyrotoxic, Myotonia, Myotonia Congenita, Narcolepsy, Neuroacanthocytosis, Neurodegeneration with Brain Iron Accumulation, Neurofi-bromatosis, Neuroleptic Malignant Syndrome, Neurological Complications of AIDS, Neurological Complications Of Lyme Disease, Neurological Consequences of Cytomega-lovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy—Hereditary, Neurosarcoidosis, Neurotoxicity, Nevus Cavernosus, Niemann-Pick Disease, Normal Pressure Hydrocephalus, Occipital Neuralgia, Occult Spinal Dysraphism Sequence, Ohtahara Syndrome, Olivopontocerebellar Atrophy, Opso-clonus Myoclonus, Orthostatic Hypotension, O'Sullivan-McLeod Syndrome, Overuse Syndrome, Pain-Chronic, Pan-tothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Postherpetic Neuralgia, Postinfectious Encephalomyelitis, Post-Polio Syndrome, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases (e.g., Creutzfeldt-Jakob disease, Gerstmann-Straussler-Scheinker syndrome, fatal familial insomnia, Kuru, scrapie or chronic wasting disease), Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudotumor Cerebri, Ramsay Hunt Syndrome I, Ramsay Hunt Syndrome II, Rasmussen's Encephalitis, Reflex Sympathetic Dystrophy Syndrome, Refsum Disease, Refsum Disease-Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizen-cephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjogren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, SUNCT Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyro toxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Tuberous Sclerosis, Vascular Erectile Tumor, Vasculitis including Temporal Arteritis, Von Economo's Disease, Von Hippel-Lindau Disease (VHL), Von Recklinghausen's Disease, Wallenberg's Syndrome, Werdnig-Hoffman Disease, Wemicke-Korsakoff Syndrome, West Syndrome, Whiplash, Whipple's Disease, Williams Syndrome, Wilson's Disease, Wolman's Disease, X-Linked Spinal and Bulbar Muscular Atrophy or Zell-weger Syndrome.

E. Combination Therapy

The compounds and compositions provided herein may also be used in combination with one or more other active ingredients. In certain embodiments, the compounds may be administered in combination, or sequentially, with another therapeutic agent. Such other therapeutic agents include those known for treatment, prevention, or amelioration of one or more symptoms associated the diseases described herein. Such therapeutic agents include, but are not limited to, agents for treatment of overweight and obesity such as appetite suppressants (e.g., phentermine, amphetamines, sibutramine/Meridia, neurotransmitter reuptake inhibitors, dopaminergic and/or serotonergic receptor agonists such as Lorcaserin; lipase inhibitors and inhibitors of fat absorption such as xenical/Orlistat and Cetilistat; regulators of energy intake), metabolism (e.g., rimonabant, taranabant and other cannabinoid receptor modulators; agents for treating metabolic syndrome including, but not limited to hyperisulinemia, insulin resistance, and diabetes such as sulfonyl ureas, meglitinide, metformin, troglitazone, pioglitazone, rosiglitazone and other PPAR modulators, and DDP-4 antagonists such as sitagliptin) agents for treating cardiovascular disease, elevated triglyceride levels, low HDL, hypercholesterolemia, and hypertension such as beta-blockers, ACE inhibitors, diuretics, nitrates, calcium channel blockers, and statins. The compounds disclosed herein may also be used in combination with dietary therapy, behavioral therapy, physical therapy, exercise, weight loss surgery, treatments for Alzheimer's Disease such as Aricept® and Excelon®; treatments for Parkinson's Disease such as L-DOPA/carbidopa, entacapone, ropinrole, pramipexole, bromocriptine, pergolide, trihexephendyl, and amantadine, agents for treating Multiple Sclerosis (MS) such as beta interferon (e.g., Avonex® and Rebif®), Copaxone®, and mitoxantrone, treatments for asthma such as albuterol and Singulair®, agents for treating schizophrenia such as zyprexa, risperdal, seroquel, and haloperidol, anti-inflammatory agents such as corticosteroids, TNF blockers, IL-1 RA, azathioprine, cyclophosphamide, and sulfasalazine, immunomodulatory and immunosuppressive agents such as cyclosporin, tacrolimus, rapamycin, mycophenolate mofetil, interferons, corticosteroids, cyclophosphamide, azathioprine, and sulfasalazine, neurotrophic factors such as acetylcholinesterase inhibitors, MAO inhibitors, interferons, anti-convulsants, ion channel blockers, riluzole and anti-Parkinsonian agents, agents for treating cardiovascular disease such as beta-blockers, ACE inhibitors, diuretics, nitrates, calcium channel blockers, and statins, agents for treating liver disease such as corticosteroids, cholestyramine, interferons and anti-viral agents, agents for treating blood disorders such as corticosteroids, anti-leukemic agents, and growth factors; and agents for treating immunodeficiency disorders such as gamma globulin.

It should be understood that any suitable combination of the compounds provided herein with one or more of the above-mentioned compounds and optionally one or more further pharmacologically active substances are considered to be within the scope of the present disclosure. In other embodiments, the compounds provided herein are administered prior to or subsequent to the one or more additional active ingredients.

F. Therapeutic Kits

Therapeutic kits comprising the compounds and compositions disclosed herein are also provided. The therapeutic kits may also contain other compounds or compositions of these other compounds.

Therapeutic kits may have a single containers which contains a compound or composition with or without other components (e.g., other compounds or compositions thereof) or may have distinct container for each component.

Therapeutic kits may include a compound and/or composition thereof packaged for use in combination with the co-administration of a second compound. The components of the kit may be pre-complexed or each component may be in a separate distinct container prior to administration to a patient.

The components of the kit may be provided in one or more liquid solutions, preferably, an aqueous solution, more preferably, a sterile aqueous solution. The components of the kit may also be provided as solids, which may be converted into liquids by addition of suitable solvents, which are preferably provided in another distinct container.

The container of a therapeutic kit may be a vial, test tube, flask, bottle, syringe, or any other means of enclosing a solid or liquid. Usually, when there is more than one component, the kit will contain a second vial or other container, which allows for separate dosing. The kit may also contain another container for a pharmaceutically acceptable liquid.

A therapeutic kit may contain apparatus (e.g., one or more needles, syringes, eye droppers, pipette, etc.), which enables administration of the components of the kit.

It will be apparent to those skilled in the art that many modifications, both to materials and methods, may be practiced without departing from the scope of the invention. Accordingly, the present embodiments are to be considered as illustrative and not restrictive, and the invention is not to be limited to the details given herein, but may be modified within the scope and equivalents of the appended claims.

The following examples are provided for illustrative purposes only and are not intended to limit the scope of the invention.

### EXAMPLES

### Example 1

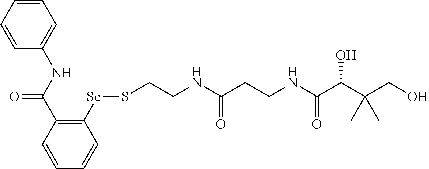

The compound above was made by reacting 2-phenyl-1,2-benzisoselenazol-3(2H)-one with D-pantetheine. 2-phenyl-1,2-benzisoselenazol-3(2H)-one (0.961 g, 3.51 mmol) was dissolved in dichloromethane (50 ml), and D-pantetheine (1 gram, 3.6 mmol) was added, followed by 20 ml methanol. The reaction was stirred under nitrogen for 16 hours, filtered through a medium glass frit and the filtrate evaporated. The residue was purified by silica gel chromatography using dichlormethane-methanol to obtain a pale yellow product (Rf=0.31 in 90:10 dichloromethane-methanol, HPLC-MS m/z=554.5). Alternatively, pantethine can be reduced to pantetheine in situ and then reacted with 2-phenyl-1,2-benzisoselenazol-3(2H)-one. For example, pantethine can be dissolved in water, treated with sodium borohydride, and then reacted with 2-phenyl-1,2-benzisoselenazol-3(2H)-one in DMSO to yield the above compound.

### Example 2

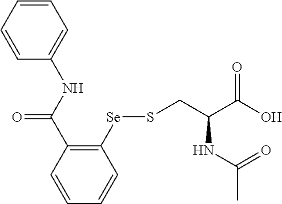

The compound above was made by dissolving 2-phenyl-1,2-benzisoselenazol-3(2H)-one (0.253 g, 0.923 mmol) in 15 ml dichloromethane under nitrogen. N-acetyl-cysteine was added, and the reaction stirred for two days. The reaction was filtered through a medium glass frit, rinsed with dichloromethane, and dried to yield a white solid (0.395 g, 0.904 mmol) characterized by HPLC-MS: m/z=439.4.

### Example 3

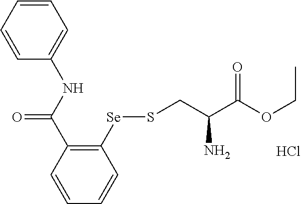

The compound above was made by dissolving 2-phenyl-1,2-benzisoselenazol-3(2H)-one (1.193 g, 4.35 mmol) in 80 ml dichloromethane and adding to H-Cys-OEt predissolved in 10 ml dichloromethane along with another 10 ml dichloromethane under nitrogen. After one hour the solvent was removed by rotary evaporation and the residue was purified by silica gel chromatography with dichloromethane-methanol. Less pure fractions were combined and evaporated to give 0.270 g of yellow syrup characterized by TLC (Rf=0.16 in 95:5 dichloromethane:methanol). The purest fractions were combined and evaporated to yield 0.943 g (2.23 mmol). This was suspended in 40 ml dichloromethane, filtered through a medium glass frit and to the filtrate was added 1 ml of 4 N HCl in dioxane. This was stirred vigorously and then filtered through a medium glass frit, rinsed with dichloromethane dried to a white solid (0.575 g, 1.25 mmol as hydrochloride salt), and characterized by HPLC-MS: m/z=425.4.

### Example 4

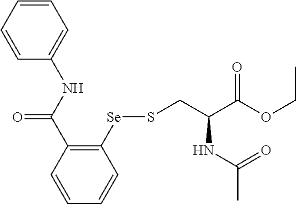

The compound above was made by dissolving material from Example 3 (0.27 g, 0.638 mmol) in 10 ml dichloromethane. To this was added para-N,N-dimethylaminopyridine (0.0419 g), 10 ml more dichloromethane, and acetic anhydride (0.07 ml, 0.741 mmol). After three days the reaction was evaporated to dryness, suspended in 25 ml water and 25 ml dichloromethane, the aqueous was extracted with another 25 ml dichloromethane, and the combined organics were filtered through a medium glass frit. The filtrate was dried over anhydrous sodium sulfate, filtered, and evaporated to a yellow film that was purified by silica gel chromatography with dichloromethane-methanol to yield an off-white foam characterized by HPLC-MS: m/z=467.4.

### Example 5

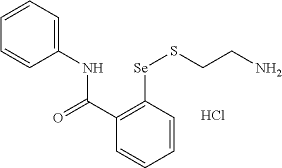

The compound above was made by dissolving 2-phenyl-1,2-benzisoselenazol-3(2H)-one (1.1643 g, 4.25 mmol) in 80 ml dichloromethane and adding cysteamine hydrochloride along with another 20 ml dichloromethane. The reaction was stirred under nitrogen for one week at which time 20 ml methanol was added and the reaction stirred another day. Then solvent was removed by rotary evaporation, the residue was resuspended in 25 ml dichloromethane, filtered through a medium glass frit and rinsed with 2×25 ml dichloromethane. The precipitate was then rinsed through the frit with methanol and evaporated to yield a yellow solid (1.55 g). A portion of this (0.577 g) was dissolved in 40 ml methanol and then 0.5 ml of 4 M HCl in dioxane was added. Solvent was removed by rotary evaporation, the residue was co-evaporated 3× from dichloromethane, and the solid was then transferred to a medium glass frit and rinsed 2× with dichloromethane to obtain 0.566 gram yellow powder. This was rinsed with 2×5 ml water and air-dried to obtain 0.519 g light yellow powder characterized by HPLC-MS: m/z=353.4.

### Example 6

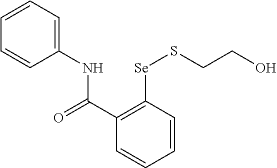

The compound above was made by dissolving 2-phenyl-1,2-benzisoselenazol-3(2H)-one (0.9934 g, 3.62 mmol) in 80 ml dichloromethane and adding 2-mercaptoethanol under nitrogen. Next day the reaction was evaporated to dryness, dissolved in dichloromethane, and purified by silica gel chromatography to yield an off-white foam (0.5 g, 1.42 mmol). Rf (95:5)=0.29, HPLC-MS: m/z=354.4.

### Example 7

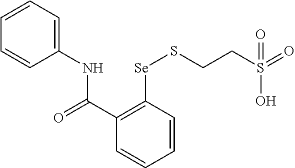

The compound above was made by dissolving 2-phenyl-1,2-benzisoselenazol-3(2H)-one (0.7156 g, 2.61 mmol) in 50 ml dry dichloromethane, dissolving sodium 2-mercaptoethanesulfonate in 20 ml methanol, and mixing. After one hour the reaction was filtered through a medium glass frit and the filtrate evaporated to dryness. A portion of this (0.304 g) was dissolved in about 30 ml of methanol, then 0.3 ml of 4 M HCl in dioxane was added. The reaction was evaporated, then coevaporated three times with dichloromethane, then resuspended in 5 ml water and filtered through a medium glass frit. The precipitate was rinsed with an additional 5 ml of water and air-dried to yield 127 mg of a white solid characterized by HPLC-MS: m/z=418.3.

### Example 8

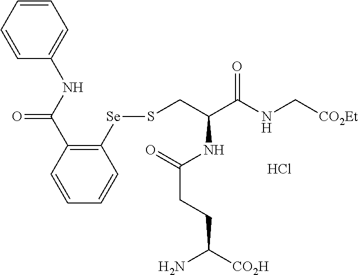

The compound above was made by dissolving 2-phenyl-1,2-benzisoselenazol-3(2H)-one (0.210 g, 0.766 mmol) in 20 ml dry dichloromethane followed by glutathione-monoethyl ester (0.249 g, 0.742 mmol) and 20 ml ethanol. The reaction was stirred under nitrogen for 90 minutes, then evaporated to dryness, resuspended in 5 ml dichloromethane, filtered through a medium glass frit, rinsed with 2×5 ml dichloromethane, and air-dried to 0.255 g of pale yellow solid. A portion of this (0.237 g) was suspended in 23 ml ethanol and 0.23 ml of 4 M HCl in dioxane was added—all dissolved. This was evaporated to dryness and then coevaporated three times from dichloromethane and dried to yield 0.219 g yellow solid characterized by HPLC-MS: m/z=611.5.

### Example 9

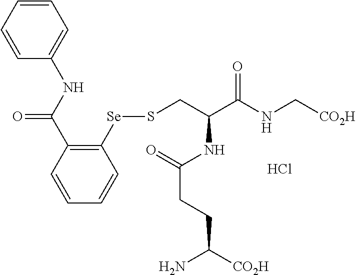

The compound above was made by dissolving 2-phenyl-1,2-benzisoselenazol-3(2H)-one (0.779 g, 2.84 mmol) in 45 ml THF followed by addition of glutathione and 10 ml more THF. The following day 25 ml ethyl acetate was added followed by 25 ml methanol. Then 4 M HCl in dioxane (1 ml) was added and the solution became transparent. The solution was evaporated to dryness, coevaporated twice from dichloromethane, transferred to a medium glass frit, and rinsed 3× with dichloromethane and dried to yield 1.747 g yellow solid characterized by HPLC-MS: m/z=583.4.

### Example 10

### RONS (Reactive Oxygen and Nitrogen Species) Interception in Cells

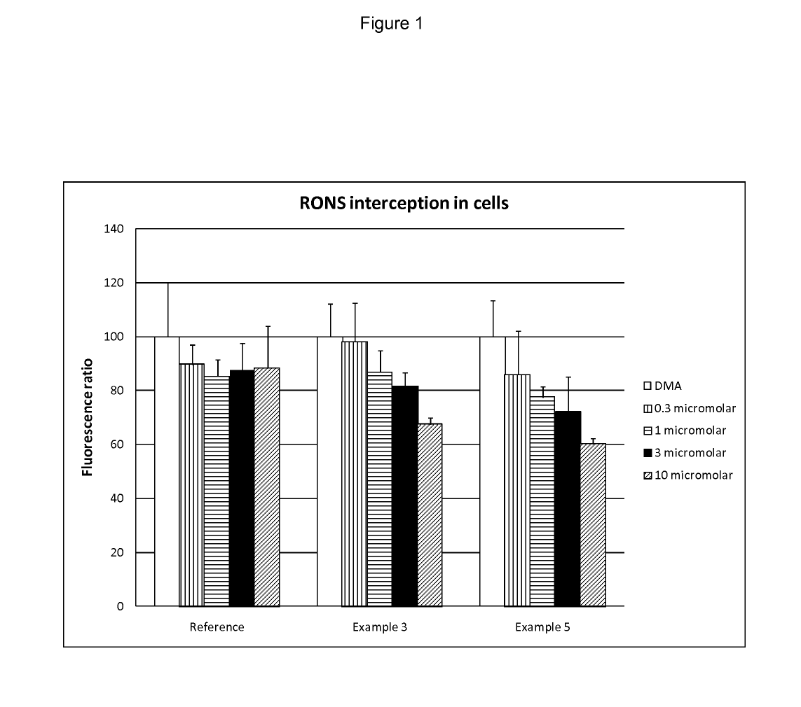

HEK293 or SY5Y cells-lines were seeded in black, clear-bottom 384-well plates at a density of 50K cells/well in media (DMEM, 10% FBS). Fluoroprobe 5-(and-6)-carboxy-2′,7′-dichlorodihydrofluorescein diacetate (carboxy-H2DCFDA) was used as an fluorescence indicator dye for the detection of intracellular oxidant species produced by metabolically stressed cells. Carboxy-H2DCFDA was prepared in dimethylformamide (DMF) at 1.8 mM and diluted to 100 μM in DPBS to generate a 10-times final stock. Cells were loaded with 10 μM Fluoroprobe for 45 minutes, 37 C. Cell plates were then washed twice with DPBS and cells were resuspended in prewarmed media (DMEM phenol red-free, 10 mM HEPES pH 7.4). Compounds were dilute in dimethylacetamide (DMA) and then diluted 1:1000 in DPBS to generate a 3-times final concentration, and administered to cells. Cell plates were read on a microplate reader immediately after compound addition to measure the baseline fluorescence intensity and then placed in a 37 C incubator. Oxidation of the fluoroprobe was detected by monitoring the increase in fluorescence with time as shown in FIG. 1. Interception of reactive oxygen and nitrogen species (RONS) in SH-SY5Y cells. Cells were loaded with a RONS sensitive fluoroprobe prior to compound treatment and induction of metabolic stress by removal of medium (t=0). Fluorescence intensity was measured at t=0, F(0), and 4.5 hours later, F(4.5), and the background signal (cells without fluoroprobe) was subtracted. The ratio of fluorescence intensity, F(4.5)/F(0), was calculated for each well and averaged for identically treated wells. For each titration series, the data was normalized with the DMA treated control set to 100. The reference compound is 2-phenyl-1,2-benzisoselenazol-3(2H)-one. The structures of the other two compounds are as shown in Example 3 and Example 5.

### Example 11

### Solubility Measurements

A 2-fold serial dilution of compounds was prepared in 25 mM Hepes pH 7.4, 100 mM NaCl. After 30 minutes at room temperature, precipitates were removed by centrifugation at 18,000×G for 15 min. The supernatant was transferred to a microtiter plate and absorbance at 260 nM was determined. The concentration at which there was no longer a linear increase in absorbance was determined as the solubility limit. The compounds in the examples generally had better solubilities than the reference compound 2-phenyl-1,2-benzisoselenazol-3(2H)-one (66-100 μM), with the compounds described in Examples 1-9 having solubilities of 300, 300, 100, 66-100, 300, 300-600, 1000, 1000, and 1000 μM.

What is claimed is:

1. A compound of Formula (I): 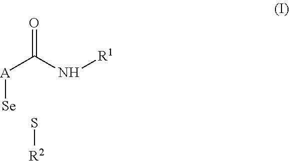
or salts, solvates or hydrates thereof, wherein:
A is aryl, substituted aryl, heteroaryl or substituted heteroaryl;
R¹ is alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R² is alkyl, substituted alkyl, heteroalkyl, substituted heteroalkyl, aryl, substituted aryl, arylalkyl, substituted arylalkyl, heteroaryl, substituted heteroaryl, heteroarylalkyl, substituted heteroarylalkyl, 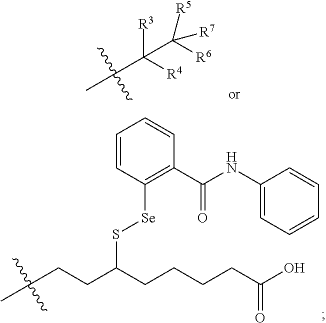
R³ is hydrogen, —OR⁹, —S(O)ₖR¹⁰, —NR¹¹R¹², —NR¹⁰COR¹³, —CO₂R¹³, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R⁵ is hydrogen, —OR¹⁴, —S(O)ₗR¹⁵, —NR¹⁶R¹⁷, —NR¹⁵COR¹⁸, —CO₂R¹⁸, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R⁷ is —CO₂R¹⁹, —CONR²⁰R²¹, —NR²²R²³, —NR²⁴COR¹⁹ or —S(O)ₘR²⁴;
R⁴, R⁶ and R⁹-R²⁴ are independently hydrogen, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl; and
k, l and m are independently 0, 1 or 2;
provided that when A and R¹ are phenyl substituted with natural amounts of deuterium that R² is not 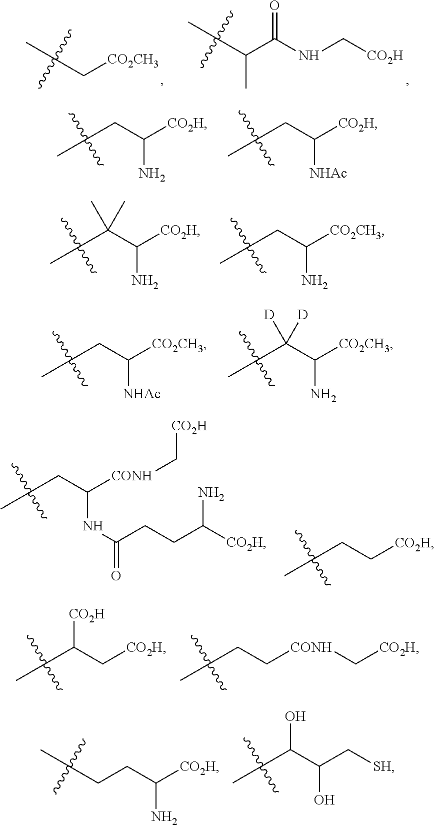
ethyl, allyl, n-propyl, isopropyl, t-butyl, n-butyl, n-hepyl, n-octyl, cyclohexyl, 4-trans-t-butylcyclohexyl, 4-cis-t-butylcyclohexyl, phenyl, p-methylphenyl, p-chlorophenyl, o-benzoic acid, o-methyl benzoate, 2-napthyl, benzyl, 2-pyridyl, 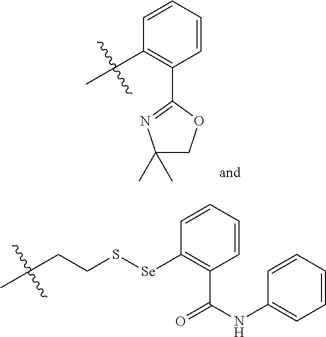
wherein the above compounds are substituted with natural amounts of deuterium, except where deuterium is explicitly specified in the structure of the above compounds

2. The compound of claim 1, wherein A is aryl, substituted aryl, phenyl or substituted phenyl;
R¹ is aryl, substituted aryl, phenyl or substituted phenyl;
R² is substituted alkyl, heteroalkyl, substituted heteroalkyl, or 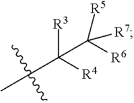
R³ is hydrogen, —OR⁹, —CO₂R¹³, alkyl or substituted alkyl;
R⁵ is hydrogen, —OR¹⁴, alkyl or substituted alkyl; and
R⁴, R⁶ and R⁹-R²⁴ are independently hydrogen, alkyl, substituted alkyl, aryl, substituted aryl, heteroaryl or substituted heteroaryl.

3. The compound of claim 1, wherein A is phenyl;
R¹ is phenyl;
R² is substituted alkyl or 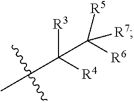
R³ is hydrogen, —OR⁹, —CO₂R¹³, alkyl or substituted alkyl;
R⁵ is hydrogen, —OR¹⁴, alkyl or substituted alkyl; and
R⁴, R⁶ and R⁹-R²⁴ are independently hydrogen, alkyl or substituted alkyl.

4. The compound of claim 1, wherein R² is 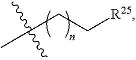
R²⁵ is —OR²⁶, —S(O)ⱼR²⁷, —NR²⁸R²⁹, —CONR²⁸R²⁹, —CO₂R³⁰, —PO(OR³¹)(OR³²), —OPO(OR³¹)(OR³²) or —NCNHR³³(NHR³⁴R³⁵) and R²⁶-R³⁴ independently hydrogen, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
n is an integer from 0 to 9; and
j is 1, 2 or 3.

5. The compound of claim 1, wherein R² is 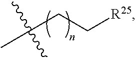
R²⁵ is —OR²⁶, —S(O)ⱼR²⁷, —NR²⁸R²⁹, —CONR²⁸R²⁹, —CO₂R³⁰, —PO(OR³¹)(OR³²), —OPO(OR³¹)(OR³²) or —NCNHR³³(NHR³⁴R³⁵), R²⁶-R²⁹ and R³¹-R³⁴ are hydrogen and R³⁰ is hydrogen, methyl or ethyl;
n is an integer from 0 to 9; and
j is 1, 2 or 3.

6. The compound of claim 1, wherein R² is 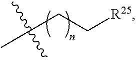
R²⁵ is —OR²⁶, —S(O)ⱼR²⁷, —NR²⁸R²⁹, —CONR²⁸R²⁹, —CO₂R³⁰, —PO(OR³¹)(OR³²), —OPO(OR³¹)(OR³²) or —NCNHR³³(NHR³⁴R³⁵), R²⁶-R²⁹ and R³¹-R³⁴ are hydrogen and R³⁰ is hydrogen, methyl or ethyl;
n is 1; and
j is 1, 2 or 3.
—CH₂CH₂CH(NHCOCH₃)CO₂H, —CH₂CH₂CH(NH₂)COCH₂CH₃. and R⁷ is —CO₂H, —CO₂CH₂CH₃, —CONHCH₂CO₂H, —CONHCH₂CO₂CH₂CH₃ or

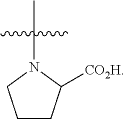

7. A compound of Formula (II): 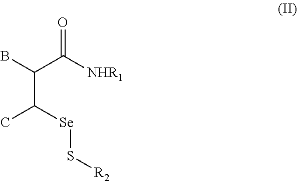
or salts, solvates or hydrates thereof, wherein:
B and C together with the atoms to which they are bonded form an aryl, substituted aryl, heteroaryl or substituted heteroaryl ring;
R¹ is alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R² is alkyl, substituted alkyl, heteroalkyl, substituted heteroalkyl, aryl, substituted aryl, arylalkyl, substituted arylalkyl, heteroaryl, substituted heteroaryl, heteroarylalkyl, substituted heteroarylalkyl, 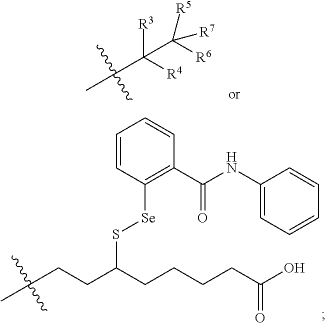
R³ is hydrogen, —OR⁹, —S(O)ₖR¹⁰, —NR¹¹R¹², —NR¹⁰COR¹³, —CO₂R¹³, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R⁵ is hydrogen, —OR¹⁴, —S(O)ₗR¹⁵, —NR¹⁶R¹⁷, —NR¹⁵COR¹⁸, —CO₂R¹⁸, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R⁷ is —CO₂R¹⁹, —CONR²⁰R²¹, —NR²²R²³, —NR²⁴COR¹⁹ or —S(O)ₘR²⁴;
R⁴, R⁶ and R⁹-R²⁴ are independently hydrogen, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl; and
k, l and m are independently 0, 1 or 2 provided that when A and R¹ are phenyl substituted with natural amounts of deuterium that R² is not 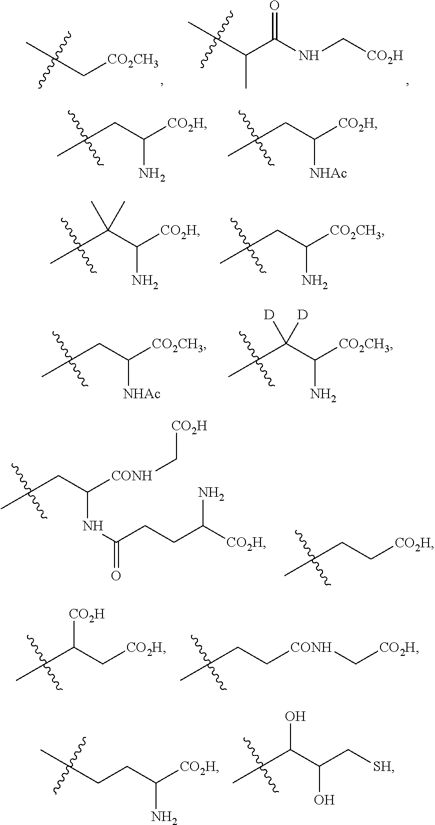
ethyl, allyl, n-propyl, isopropyl, t-butyl, n-butyl, n-hepyl, n-octyl, cyclohexyl, 4-trans-t-butylcyclohexyl, 4-cis-t-butylcyclohexyl, phenyl, p-methylphenyl, p-chlorophenyl, o-benzoic acid, o-methyl benzoate, 2-napthyl, benzyl, 2-pyridyl, 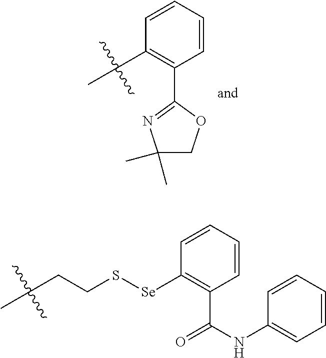
wherein the above compounds are substituted with natural amounts of deuterium, except where deuterium is explicitly specified in the structure of the above compounds.

8. The compound of claim 7, wherein B and C form an aryl, substituted aryl, phenyl or substituted phenyl ring;
R¹ is aryl, substituted aryl, phenyl or substituted phenyl;
R² is substituted alkyl, heteroalkyl, substituted heteroalkyl, or 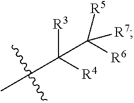
R³ is hydrogen, —OR⁹, —CO₂R¹³, alkyl or substituted alkyl;
R⁵ is hydrogen, —OR¹⁴, alkyl or substituted alkyl; and
R⁴, R⁶ and R⁹-R²⁴ are independently hydrogen, alkyl, substituted alkyl, aryl, substituted aryl, heteroaryl or substituted heteroaryl.

9. The compound of claim 7, wherein B and C form a phenyl ring;
R¹ is phenyl;
R² is substituted alkyl, or 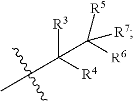
R³ is hydrogen, —OR⁹, —CO₂R¹³, alkyl or substituted alkyl;
R⁵ is hydrogen, —OR¹⁴, alkyl or substituted alkyl; and
R⁴, R⁶ and R⁹-R²⁴ are independently hydrogen, alkyl or substituted alkyl.

10. The compound of claim 7, wherein R² is 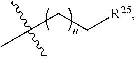
R²⁵ is, —OR²⁶, —S(O)ⱼR²⁷, —NR²⁸R²⁹, —CONR²⁸R²⁹, —CO₂R³⁰, —PO(OR³¹)(OR³²), —OPO(OR³¹)(OR³²) or —NCNHR³³(NHR³⁴R³⁵) and R²⁶-R³⁴ are independently hydrogen, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
n is an integer from 0 to 9; and
j is 1, 2 or 3.

11. The compound of claim 7, wherein R² is 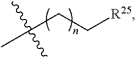
R²⁵ is —OR²⁶, —S(O)ⱼR²⁷, —NR²⁸R²⁹, —CONR²⁸R²⁹, —CO₂R³⁰, —PO(OR³¹)(OR³²), —OPO(OR³¹)(OR³²) or —NCNHR³³(NHR³⁴R³⁵), R²⁶-R²⁹ and R³¹-R³⁴ are hydrogen and R³⁰ is hydrogen, methyl or ethyl;
n is an integer from 0 to 9; and
j is 1, 2 or 3.

12. The compound of claim 7, wherein R² is 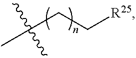
R²⁵ is, —OR²⁶, —S(O)ⱼR²⁷, —NR²⁸R²⁹, —CONR²⁸R²⁹, —CO₂R³⁰, —PO(OR³²), —OPO(OR³¹)(OR³²) or —NCNHR³³(NHR³⁴R³⁵), R²⁶-R²⁹ and R³¹-R³⁴ are hydrogen and R³⁰ is hydrogen, methyl or ethyl;
n is 1 and
j is 1, 2 or 3.

13. A compound of Formula (III): 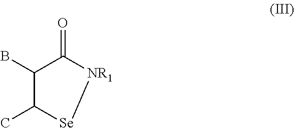
or salts, solvates or hydrates thereof, wherein:
B and C together with the atoms to which they are bonded form an aryl, substituted aryl, heteroaryl or substituted heteroaryl ring;
R¹ is alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
wherein at least one hydrogen in Formula (III) is substituted with deuterium.

14. A composition comprising a compound of Formula (II): 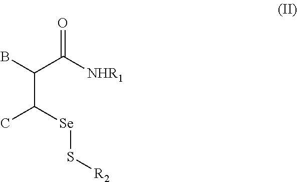
or salts, solvates or hydrates thereof, wherein:
B and C together with the atoms to which they are bonded form an aryl, substituted aryl, heteroaryl or substituted heteroaryl ring;
R¹ is alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R² is alkyl, substituted alkyl, heteroalkyl, substituted heteroalkyl, aryl, substituted aryl, arylalkyl, substituted arylalkyl, heteroaryl, substituted heteroaryl, heteroarylalkyl, substituted heteroarylalkyl, 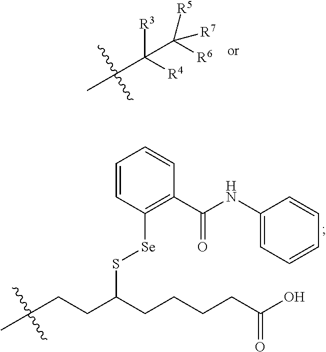
R³ is hydrogen, —OR⁹, —S(O)ₖR¹⁰, —NR¹¹R¹², —NR¹⁰COR¹³, —CO₂R¹³, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R⁵ is hydrogen, —OR¹⁴, —S(O)ₗR¹⁵, —NR¹⁶R¹⁷, —NR¹⁵COR¹⁸, —CO₂R¹⁸, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R⁷ is —CO₂R¹⁹, —CONR²⁰R²¹, —NR²²R²³, —NR²⁴COR¹⁹ or —S(O)ₘR²⁴;
R⁴, R⁶ and R⁹-R²⁴ are independently hydrogen, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl; and
k, l and m are independently 0, 1 or 2 and a vehicle.

15. A method of treating an addictive disorder, obesity, skin disorder, lung disorder or respiratory disease, neuropsychiatric disorder, traumatic injury, ischemia or ischemia-reperfusion injury, ocular disease, a neurological disorder or a neurodegenerative disease in a patient comprising administering to the patient in need thereof a compound of Formula (II): 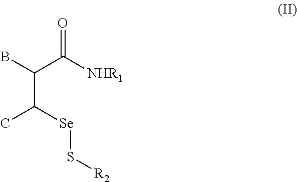
or salts, solvates or hydrates thereof, wherein:
B and C together with the atoms to which they are bonded form an aryl, substituted aryl, heteroaryl or substituted heteroaryl ring;
R¹ is alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R² is alkyl, substituted alkyl, heteroalkyl, substituted heteroalkyl, aryl, substituted aryl, arylalkyl, substituted arylalkyl, heteroaryl, substituted heteroaryl, heteroarylalkyl, substituted heteroarylalkyl, 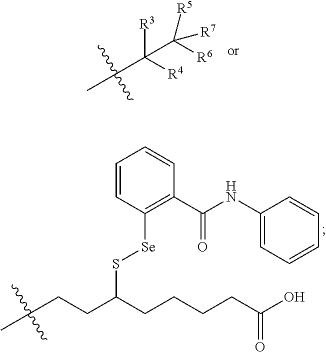
R³ is hydrogen, —OR⁹, —S(O)ₖR¹⁰, —NR¹¹R¹², —NR¹⁰COR¹³, —CO₂R¹³, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R⁵ is hydrogen, —OR¹⁴, —S(O)ₗR¹⁵, —NR¹⁶R¹⁷, —NR¹⁵COR¹⁸, —CO₂R¹⁸, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R⁷ is —CO₂R¹⁹, —CONR²⁰R²¹, —NR²²R²³, —NR²⁴COR¹⁹ or —S(O)ₘR²⁴;
R⁴, R⁶ and R⁹-R²⁴ are independently hydrogen, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl; and
k, l and m are independently 0, 1 or 2 or the composition of claim 14.

16. A composition comprising the compound of claim 13 and a vehicle.

17. A method of treating an addictive disorder, obesity, skin disorder, lung disorder or respiratory disease, neuropsychiatric disorder, traumatic injury, ischemia or ischemia-reperfusion injury, ocular disease, a neurological disorder or a neurodegenerative disease in a patient comprising administering to the patient in need thereof the compound of claim 13 or the composition of claim 16.

18. A composition comprising the compound of Formula (I): 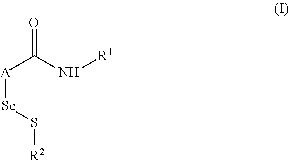
or a salt, solvate or hydrate thereof, wherein:
A is aryl, substituted aryl, heteroaryl or substituted heteroaryl;
R¹ is alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R² is alkyl, substituted alkyl, heteroalkyl, substituted heteroalkyl, aryl, substituted aryl, arylalkyl, substituted arylalkyl, heteroaryl, substituted heteroaryl, heteroarylalkyl, substituted heteroarylalkyl, 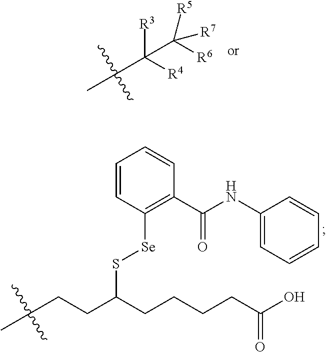
R³ is hydrogen, —OR⁹, —S(O)ₖR¹⁰, —NR¹¹R¹², —NR¹⁰COR¹³, —CO₂R¹³, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R⁵ is hydrogen, —OR¹⁴, —S(O)ₗR¹⁵, —NR¹⁶R¹⁷, —NR¹⁵COR¹⁸, —CO₂R¹⁸, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R⁷ is —CO₂R¹⁹, —CONR²⁰R²¹, —NR²²R²³, —NR²⁴COR¹⁹ or —S(O)ₘR²⁴;
R⁴, R⁶ and R⁹-R²⁴ are independently hydrogen, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl; and
k, l and m are independently 0, 1 or 2; and
a vehicle.

19. A method of treating an addictive disorder, obesity, skin disorder, lung disorder or respiratory disease, neuropsychiatric disorder, traumatic injury, ischemia or ischemia-reperfusion injury, ocular disease, a neurological disorder or a neurodegenerative disease comprising administering to a patient in need thereof a compound of Formula (I): 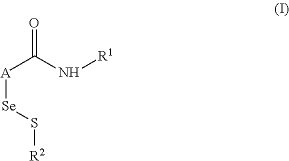
or a salt, solvate or hydrate thereof, wherein:
A is aryl, substituted aryl, heteroaryl or substituted heteroaryl;
R¹ is alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R² is alkyl, substituted alkyl, heteroalkyl, substituted heteroalkyl, aryl, substituted aryl, arylalkyl, substituted arylalkyl, heteroaryl, substituted heteroaryl, heteroarylalkyl, substituted heteroarylalkyl, 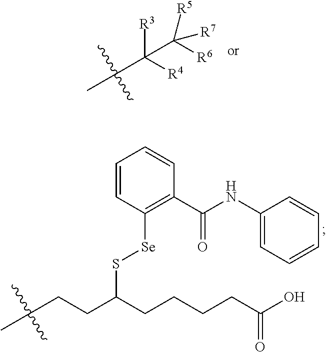
R³ is hydrogen, —OR⁹, —S(O)ₖR¹⁰, —NR¹¹R¹², —NR¹⁰COR¹³. —CO₂R¹³, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R⁵ is hydrogen, —OR¹⁴, —S(O)ₗR¹⁵, —NR¹⁶R¹⁷, —NR¹⁵COR¹⁸, —CO₂R¹⁸, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl;
R⁷ is —CO₂R¹⁹, —CONR²⁰R²¹, —NR²²R²³, —NR²⁴COR¹⁹ or —S(O)ₘR²⁴;
R⁴, R⁶ and R⁹-R²⁴ are independently hydrogen, alkyl, substituted alkyl, heteroalkyl, substituted heteroalkyl, aryl, substituted aryl, arylalkyl, substituted arylalkyl, heteroaryl, substituted heteroaryl, heteroarylalkyl or substituted heteroarylalkyl; and
k, l, m are independently 0, 1 or 2 or the composition of claim 17.
